(19) United States
(12) Patent Application Publication
(10) Pub. No.: US 2024/0050582 A1
(43) Pub. Date: Feb. 15, 2024

(54) CONJUGATES INCLUDING MULTIPLE SACCHARIDIC CHAINS ON A LINEAR PROTEIN AND USES IN MAMMALS FEED

(71) Applicants: UNIVERSITEIT GENT, GENT (BE); ELICITYL, CROLLES (FR)

(72) Inventors: Eric COX, OOSTERZELE (BE); Ut NGUYEN VAN, ZWIJNAARDE (BE); Benoit DARBLADE, CROLLES (FR); Ludovic BASTIDE, LES ADRETS (FR); Silvère BONNET, GRENOBLE (FR)

(21) Appl. No.: 18/280,316

(22) PCT Filed: Mar. 7, 2022
(86) PCT No.: PCT/EP2022/055763

§ 371 Date: Sep. 5, 2023

(30) Foreign Application Priority Data
Mar. 8, 2021 (EP) 21305277.2

(51) Int. Cl. A61K 47/64; A61P 31/04

(52) U.S. Cl. CPC A61K 47/6455; A61P 31/04

(57) ABSTRACT

Conjugates (I), useful for pig feeds, include saccharidic chains X(Fucα2)Galβk(Y)TV (II) covalently grafted on a linear protein W, wherein: X is absent, or is Galα3 or GalNAcα3 and when X is absent, then Y is absent; Y is absent or is Fucα2 or Fucα4; T is absent or is ZNAcε3; wherein Z is Glc or Gal; and ε is α or β; V is absent or is a mono, or an oligosaccharide; n is an integer equal to 8 or more, k is 3 or 4, and wherein the covalent grafting occurs either between V and the linear protein W, when V is present, or between T and the linear protein W, when T is present and V is absent, or between Galβk and the linear protein W, when both T and V are absent; and wherein the saccharidic unit covalently bonded to the linear protein may be open and acyclic.

### FIELD OF THE INVENTION

The present invention relates to new oligosaccharide conjugates or polysaccharide conjugates and the use thereof for preventing and treating F18⁺ Escherichia coli infections in mammals. In particular, according to the invention the oligosaccharide or a polysaccharide is covalently grafted on a linear protein.

### BACKGROUND OF THE INVENTION

Enterotoxigenic (ETEC) and verotoxigenic (VTEC) Escherichia coli (E. coli) which are F18⁺ Escherichia coli are important causes of disease in man and animal.

As explained in WO 2010/037785, by adhering to the mucosa, bacteria are prevented from being eradicated by the natural cleaning mechanisms of the host such as intestinal peristaltism and secretion of fluid. Furthermore, by being in close contact with the mucosal surfaces, the bacteria have better access to the available nutrients and the secreted toxins are delivered close to their target tissue. Attachment to host tissues is mediated by adhesins expressed on the surface of the bacteria. The bacterial adhesins are known to be lectins that combine with complementary carbohydrates on the host cell surfaces. The bacterial adhesins are part of typical polymeric structures that are named fimbriae or pili. Diseases, such as diarrhea and/or edema disease, frequently can occur in pigs shortly after their weaning. Main causes are ETEC or VTEC E. coli expressing F18 fimbriae. F18 fimbriae are expressed by the fed (fimbriae associated with edema disease) gene cluster and are typically composed of multiple copies of the major subunit FedA whereas a minor subunits FedF is only present in small amounts. FedF was determined to be the adhesive subunit.

F18⁺ E. coli use their fimbriae to attach to specific receptors on the pig intestinal epithelium and produce enterotoxins (LT, STa and/or STb) and/or verotoxins (VTx2e) leading to diarrhea or edema disease, respectively.

Infections lead to considerable economic losses due to mortality, decreased growth rate and cost of medication. Antibiotics are routinely used to combat these infections, but due to the emergence of antimicrobial resistance, there is an urgent need for alternatives. Previously, it was shown that F18⁺ E. coli specifically interacts with blood group ABH determinants on type 1 core chains (WO 2010/037785). So, WO 2010/037785 provides the use of a compound for binding F18⁺ E. coli, F18 fimbriae, F18 adhesin, FedF or the receptor binding of FedF of formula:

[X(Fucα2)Galβ3(Y)TV]ₙ-W
wherein
X is absent, Galα3 or GalNAcα3 and when X is absent, then Y is absent;
Y is absent or Fucα4;
T is absent or ZNAcε3;
and wherein Z is Glc or Gal; and ε is α or β
V is absent or a mono- or polysaccharide;
n is 1 or more; and
W is absent or a carrier capable of binding n chains X(Fucα2)Galβ3(Y)TV.

The carrier can be a mono- or polysaccharide, a protein, a lipid, a glycolipid, a glycoprotein, a glycosphingolipid, a ceramide, lectins, antibodies, immunoglobulines, synthetic mimics of the aforementioned carriers, organic molecules, small molecules, chemicals, nanoparticles, beads, gels.

In the examples of WO 2010/037785, only non-conjugated oligosaccharides or oligosaccharides conjugated to human serum albumin carrier (HSA) are studied.

Based on this, an anti-adhesive therapy was considered. A high dose of the monomeric compounds could inhibit in vitro the binding of F18⁺ E. coli to porcine intestinal villi. In addition, multimerization of the monomeric receptor structure on a human serum albumin carrier (HSA) could significantly enhance efficacy at high concentrations. Multimerization of saccharides has been described earlier for different applications (Miura et al. 2015) and a plethora of carrier structures is available. However, whether inhibition benefits from multimer systems depends on the spatial orientation of the oligosaccharides, the density of grafting of the oligosaccharides on the carrier and the degree of polymerization of the carrier.

Feed additive companies are highly interested in bringing alternatives of antibiotics to the market, but no feed alternative that is currently available can claim to be effective against (E. coli) diarrhoea and cannot show in vivo effectiveness. In addition and in view of economic reasons, oligo or polysaccharide concentrations should be as low as possible and effective over a wide dosage range in order to be suitable as feed additive.

Therefore, it was the aim of the present invention to identify new multimeric forms of poly or oligosaccharides that are safe to be used in the food chain and can be easily produced.

### SUMMARY OF THE INVENTION

The invention proposes conjugates (I) which comprise n saccharidic chains X(Fucα2)Galβk(Y)TV (II) covalently grafted on a linear protein W, wherein:
X is absent, or is Galβ3 or GalNAcα3 and when X is absent, then Y is absent;
Y is absent or is Fucα2 or Fucα4;
T is absent or is ZNAcε3;
and wherein Z is Glc or Gal; and ε is α or β;
V is absent or is a mono, or an oligosaccharide;
n is an integer equal to 8 or more;
k is 3 or 4;
   and wherein the covalent grafting occurs either between V and the linear protein W, when V is present, or between T and the linear protein W, when T is present and V is absent, or between Galβk and the linear protein W, when both T and V are absent.

In particular, in the conjugates (I) according to the invention, k is 3.

In particular, in the conjugates (I) according to the invention, W is a polylysine, in particular a poly-L-lysine, such as ε-poly-L-lysine.

According to some embodiments of the conjugates (I) of the invention, the saccharidic chains (II) are covalently coupled on the linear protein, by the saccharidic unit corresponding to their reducing end which is open and so acyclic and has been coupled by its aldehyde function to a primary amino function of the linear protein by reductive amination.

According to interesting embodiments, in the conjugates (I) of the invention, n is in the range from 8 to 240, for instance from 8 to 100, from 8 to 50, particularly from 8 to 40 and in particular in the range from 10 to 35, more specifically in the range from 12 to 35.

In particular, in the conjugates (I) of the invention, X is absent and Y is absent, then T is absent or is ZNAcε3, with Z is Glc.

According to particular embodiments, which may be combined to the previous ones, in the conjugates (I) of the invention, X is Galα3 or GalNAcα3; Z is Glc or Gal and Y is absent.

In particular conjugates (I) of the invention, the saccharidic chains (II) have the formula (IIa):

X(Fucα2)Galβ3(Y)ZNAcε3UGalβ4Glc (IIa)
wherein U is absent, is Galα4, Galβ3GlcNAcβ3, or (Fucα2)Galβ3GlcNAcβ3 and ε, X, Y and Z are as defined herein.

According to the invention, the conjugates (I) comprise n saccharidic chains consisting of formula X(Fucα2)Galβk(Y)TV (II) or consisting of formula X(Fucα2)Galβ3(Y)ZNAcε3UGalβ4Glc (IIa), covalently grafted on a linear protein W, with n, X, k, Y, T, V, E, Z and U as defined herein.

In particularly interesting conjugates (I), the saccharidic chains (II) are selected from the group consisting of the following oligosaccharides:
Fucα2Gal;
Fucα2Galβ3GlcNAcβ3Gal;
Fucα2Galβ3GlcNAcβ3Galβ4Glc (H5-1);
Galα3(Fucα2)Galβ3GlcNAcβ3Gal;
Galα3(Fucα2)Gap 3GlcNAcβ3Galβ4Glc (B6-1);
GalNAcα3(Fucα2)Gal;
GalNAcα3(Fucα2)Galβ4Glc (A4-5);
GalNAcα3(Fucα2)Galβ3GlcNAcβ3Gal (A5-1);
GalNAcα3(Fucα2)Galβ3GlcNAcβ3Galβ4Glc (A6-1);
GalNAcα3(Fucα2)Galβ4GlcNAcβ3Galβ4Glc (A6-2);
GalNAcα3(Fucα2)Galβ3(Fucα4)GlcNAcβ3Gal;
GalNAcα3(Fucα2)Galβ3(Fucα4)GlcNAcβ3Galβ4Glc;
Galα3(Fucα2)Galβ3(Fucα4)GlcNAcβ3Gal;
Galα3(Fucα2)Galβ3(Fucα4)GlcNAcβ3Galβ4Glc;
GalNAcα3(Fucα2)Galβ3GlcNAcβ3Galβ3GlcNAcβ3Gal;
GalNAcα3(Fucα2)Galβ3GlcNAcβ3Galβ3GlcNAcβ3Galβ4Glc;
GalNAcα3(Fucα2)Galβ3GalNAcβ3Galα4Gal;
GalNAcα3(Fucα2)Galβ3GalNAcβ3Galα4Galβ4Glc;
GalNAcα3(Fucα2)Galβ3GalNAcα3(Fucα2)Galβ3GlcNAcβ3Gal and
GalNAcα3(Fucα2)Galβ3GalNAcα3(Fucα2)Galβ3GlcNAcβ3Galβ4Glc.

According to particular embodiments, which may be combined to the previous ones, the conjugates (I) of the invention comprise polylysine, and in particular, ε-poly-L-lysine, as linear protein, which has a weight average molecular weight Mw in the range from 2000 to 33000 g/moL, in particular in the range from 3200 to 6850 g/moL and/or an average degree of polymerization (DP) in the range from 15 to 240, in particular in the range from 20 to 50.

According to particular embodiments, which may be combined to the previous ones, the conjugates (I) of the invention have a weight average molecular weight Mw in the range from 5000 to 410000 g/moL, in particular in the range from 5600 to 86000 g/moL.

The invention also provides pharmaceutical compositions comprising a pharmaceutically acceptable carrier and one or more of the conjugates (I) according to the invention in a therapeutically effective amount.

A food or drink supplemented with one or more of the conjugates (I) of the invention are another object of the invention.

In particular, in such a drink, the quantity of said conjugate(s) represents 0.05 g/100 L to 5 g/100 L, in particular 0.1 g/100 L to 2 g/100 L mg/L of the said drink.

The invention also provides a pig feed composition supplemented with one or more of the conjugates (I) of the invention.

In particular, in such a food or in such a pig feed, the quantity of said conjugate(s) represents 5 mg/kg to 50 mg/kg, in particular 8 mg/kg to 20 mg/kg of the said food or pig feed.

The invention also concerns the conjugates (I) according to the invention, for use as a medicament.

According to particular embodiments, the conjugates (I) according to the invention are for use in treating or preventing F18⁺ E. coli infections in mammals, such as pigs, in particular in treating or preventing of a post weaning diarrhea and/or edema disease in pigs.

According to particular embodiments, the conjugate (I) for use according to the invention binds to F18⁺ E. coli, F18 fimbriae, F18 adhesin, FedF or to the receptor binding domain of FedF.

According to particular embodiments, the conjugate (I) for use according to the invention is administered orally, in particular is included in food or drink.

According to particular embodiments, the conjugates (I) of the invention are for use on pigs and the conjugate is included in a pig feed composition.

The invention also provides a method for preparing a conjugate (I) according to the invention wherein n molecules of saccharidic chains X(Fucα2)Galβk(Y)TV (II), with n, k, X, Y, T and V as defined herein, are grafted by covalent coupling, via their reduced end on a molecule of a linear protein W.

According to particular embodiments of this method, the grafting is obtained by reaction between the oligosaccharide or polysaccharide of formula X(Fucα2)Galβk(Y)TV (II) with n, k, X, Y, T and V as defined herein and the linear protein.

In particular, the linear protein has pendant primary amino groups, in particular is polylysine, and the grafting on the linear protein is carried out by reaction of reductive amination, between primary amino groups of the linear protein, in particular polylysine, and the aldehyde group of the reducing-end in its acyclic form of the oligosaccharide or polysaccharide of formula X(Fucα2)Galβk(Y)TV (II), with n, k, X, Y, T and V as defined herein, in presence of a reducing agent, such as NaBH₃CN.

### BRIEF DESCRIPTION OF THE DRAWINGS

FIG. 1: In situ activity of A6-1-PL in the segment perfusion test, 1=F4+ toxin negative control strain and gives background fluid absorption in presence of a non-pathogenic E. coli strain; 2 to 7 receive different concentrations of Coligo; 8 receives the polylysine carrier.

FIG. 2: Comparison of different A6-1 conjugates and their carriers for inhibition of the binding of F18⁺ E. coli to villi. Different carriers were used for multimerization of blood group sugar A6 type 1 and compared for specific inhibition of binding of F18⁺ E. coli to the villi of F18R+ piglets. Monomeric A6-1 10 mg/ml was a positive control. Only the polylysine A6-1 multimer specifically reduced adhesion for actual concentrations of A6-1 between 1.2 and 0.12 μg:ml since the unconjugated polylysine showed no inhibitory effect. Pectin, CMCL, CMCM gave strong background (as was the case for the non-shown carriers alginate, methylacrylate, micronized cellulose). Polylysin shows low background.

FIG. 3: Increasing concentrations of A6-1 polylysine multimer results in aspecific inhibition of binding of F18 E. coli to villi by polylysine in the in vitro inhibition test. The concentrations in the X-ax present the actual concentration of A6-1 in the multimer. Different concentrations of A6-1 PL are evaluated and demonstrate that low concentrations give important inhibition.

FIG. 4: CMCL and CMCM show an A6-1 independent inhibition of binding of the bacteria to villi and PL does not. The inhibition seen with the high concentrations of PL, can be removed by washing bacteria before incubation. The A6-1 PL conjugate is not removed by washing which supports specificity of this interaction.

FIG. 5: Mean relative percent of inhibition of binding of F18 E. coli to villi±SEM by A6-1 multimers of blood group A hexaose type 1 with different carriers. The blackline presents background inhibition. Protein carriers for A6-1 and a low and high conjugation rate for these carriers are compared. A6-1 PL gives the best inhibition at low concentrations.

FIG. 6: Blood group A6 type 1 conjugated to polylysine showed the most consistent inhibition of binding of F18⁺ E. coli to villi of F18 R+ pigs. The figure presents inhibition of binding by polylysine, the monomers A6-1, H5-1 and LNT and the conjugates of A6-1, A4-5, H5-1, LNT and a MIX of A6-1 (85%) and H5-1+LNT (10%) using polylysine as carrier. The blackline presents background inhibition. The conjugate of A6-1 with PL showed the most consistent inhibition at low concentrations.

FIG. 7: Comparing area under the curve of F18⁺ E. coli shedding between groups. The area represents the effect of the treatment using A6-1 PL on the colonization of F18⁺ E. coli in the gut during the whole 15-day duration. The asterisk indicates that the area is significant different in comparison with the control group (for both p=0.03).

FIG. 8: Mean shedding duration of drinking water (9 days) and feed group (10 days) were significantly shorter than this of the control (15 days) (p=0.0035 and 0.0034, respectively).

### DETAILED DESCRIPTION

### Definitions

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

Unless otherwise stated, any numerical values, such as a concentration or a concentration range described herein, could be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% includes 0.9% to 11%.

Nevertheless, the specific mentioned value is always preferred. As used herein, the use of a numerical range expressly includes all possible subranges, all individual numerical values within that range, including integers within such ranges and fractions of the values unless the context clearly indicates otherwise.

As used herein, the terms “comprises,” “comprising,” “includes”, “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, or a process that comprises a list of elements is not necessarily limited to only those elements but can include other elements not expressly listed or inherent to such composition or method.

As used herein, the term “consists of,” or variations such as “consist of or “consisting of,” as used throughout the specification and claims, indicate the inclusion of any recited integer or group of integers, but that no additional integer or group of integers can be added to the specified method, structure, or composition.

Unless otherwise stated, “conjugate” or “conjugates” in the specification, designates any conjugate (I) as defined in the invention, so, also any more specific conjugates described in the invention. As the conjugates of the invention comprise several molecules of oligo or polysaccharide, they are also named multimers.

As used herein, the term “consists essentially of,” or variations such as “consist essentially of or “consisting essentially of,” as used throughout the specification and claims, indicate the inclusion of any recited integer or group of integers, and the optional inclusion of any recited integer or group of integers that do not materially change the basic or novel properties of the specified method, structure or composition.

### Problem Solved by the Invention

Previous research has shown that particular blood group oligosaccharides encompassed by the formula (II) can specifically inhibit the adhesion of F18+ E. coli to porcine intestinal villi in vitro. In the present invention, several conjugates of these oligosaccharides were constructed and compared. Specific types of conjugates on linear protein were identified, as being very efficient, by comparison to the others.

They were able to decrease specifically binding in vitro with more than 40% decrease, and this at concentrations which are more than 100 times lower, than the concentration of the monomeric oligosaccharide, and approximately 10 times lower than the concentration of other carrier-type based conjugates. The novel conjugates according to the invention were shown to prevent fluid loss (a measure of diarrhea) due to inoculation with F18+ STa+STb+ ETEC in situ in perfused small intestinal segments and significantly decrease duration and height of excretion of F18ab+ VTEC in vivo in newly weaned piglets, this, remarkably, at concentrations below or equal to 8 μg/g in the pig's diet. One of the most efficient conjugates according to the invention was also successfully tested in vivo.

A major advantage of the use of the conjugates of the present invention is the ease of administration via food or drinking water and the broad activity against F18⁺ ETEC and VTEC strains.

Conjugates

The present invention provides conjugates comprising a linear protein which carries several molecules of oligosaccharide or polysaccharide. These conjugates bind to F18⁺ E. coli, F18 fimbriae, F18 adhesin, FedF or the receptor binding domain of FedF.

More specifically, the invention provides conjugates (I) which comprise n saccharidic chains X(Fucα2)Galβk(Y)TV (II) covalently grafted on a linear protein W, wherein:
X is absent, or is Galα3 or GalNAcα3 and when X is absent, then Y is absent;
Y is absent or is Fucα2, or preferably Fucα4;
T is absent or is ZNAcε3;
and wherein Z is Glc or Gal; and ε is α, or preferably β;
V is absent or is a mono, or an oligosaccharide;
n is an integer equal to 8 or more;
k is 4, or preferably 3;
and wherein the covalent grafting occurs either between V and the linear protein W, when V is present, or between T and the linear protein W, when T is present and V is absent, or between Galβk and the linear protein W, when both T and V are absent.

An oligosaccharide contains three to ten monosaccharide units. So, according to the definition of V, the saccharidic chain X(Fucα2)Galβk(Y)TV (II), with n, k, X, Y, T and V as defined herein, may be an oligosaccharide or a polysaccharide which includes more than ten monosaccharide units. In particular, X(Fucα2)Galβk(Y)TV (II) is an oligosaccharide. In the specification, X(Fucα2)Galβk(Y)TV (II), with n, k, X, Y, T and V as defined herein, is simply named saccharidic chain.

“conjugates (I) which comprise n saccharidic chains X(Fucα2)Galβk(Y)TV (II) covalently grafted on a linear protein” means that n molecules of X(Fucα2)Galβk(Y)TV (II) are directly or indirectly attached by a covalent bond to one molecule of linear protein W or than n saccharidic chains obtained from X(Fucα2)Galβk(Y)TV (II) after a grafting step are attached by a covalent bond to one molecule of linear protein W. So, the “covalently grafted saccharidic chains X(Fucα2)Galβk(Y)TV (II)” includes:
when both T and V are absent: X(Fucα2)Galβ- or X(Fucα2)Gal′-, wherein Gal′ is Gal which has been modified by the grafting step,
when V is absent and T is present: X(Fucα2)Galβk(Y)T- or X(Fucα2)Galβk (Y)T′-, wherein T′ is T which has been modified by the grafting step,
when V is present: X(Fucα2)Galβk(Y)TV- or X(Fucα2)Galβk(Y)TV′-, wherein V′ is V which has been modified by the grafting step.

In other words, the conjugates (I) of the invention can be defined, as being obtained by a covalent grafting of n saccharidic chains X(Fucα2)Galβk(Y)TV (II), on a linear protein W. The conjugates (I) of the invention may be obtained by coupling n molecules of oligosaccharide or polysaccharide of formula X(Fucα2)Galβk(Y)TV (II), with a (non-grafted) linear protein W to obtain a covalent attachment. In that case, the grafting involves a reaction between the oligosaccharide or polysaccharide of formula X(Fucα2)Galβk(Y)TV (II) and the linear protein. So, the saccharidic chains X(Fucα2)Galβk(Y)TV (II) may have been slightly modified by the grafting operation: in particular, the saccharidic unit of its reducing end (namely terminal end of V when V is present, or terminal end of T when T is present and V is absent, or Galβk, when both T and V are absent) may be acyclic and bond to the linear protein in the obtained conjugate. So, “saccharidic chains X(Fucα2)Galβk(Y)TV (II) covalently grafted” encompasses such modified saccharidic chains. This grafting may lead to the denaturation of the saccharidic unit which is present at the reducing end of the oligo or polysaccharide of formula X(Fucα2)Gal(k(Y)TV (II). In particular, the saccharidic unit which is present at the reducing end of the saccharidic chains may be in its acyclic form and can have lost a part of its structure, during the grafting, and so may be altered in the conjugate. This will be explained in details, in the specification hereafter. In particular, when the covalent bond of the conjugate between the linear protein and its saccharidic part (named saccharidic chains X(Fucα2)Galβk(Y)TV (II) covalently grafted) is obtained via a reductive amination with free primary amino functions —NH₂ available on the (ungrafted) linear protein, the reaction of the aldehyde function of the reducing end of the oligosaccharide or polysaccharide X(Fucα2)Galβk(Y)TV (II) and these primary amino functions —NH₂ leads to a —CH2-NH— link, as detailed hereafter. It is also possible that the grafting involves a reaction between the (ungrafted) linear protein and saccharidic chains X(Fucα2)Galβk(Y)TV (II) previously modified for including a reactive function, potentially with the presence of a spacer arm. In that case, in the conjugates of the invention, the saccharidic chains X(Fucα2)Galβk(Y)TV (II) may be present, as such, and covalently bound to the linear protein, potentially via a spacer arm. Whatever the way of obtaining the conjugates of the invention, the covalent grafting on the linear protein W occurs:
either by the intermediary of V, when V is present, and may be obtained via a spacer arm;
or by the intermediary of T, when T is present and V is absent, and may be obtained via a spacer arm;
or by the intermediary of Galβk, when both T and V are absent, and may be obtained via a spacer arm.

“Linear protein” means that the structure of the (ungrafted) protein is linear and so corresponds to a linear chain without intra-chain cross-link (i.e. without intra-chain covalent bond). So, this not excludes the fact that the protein may adopt different conformations (linear or not), especially in water or another solvent. The linear protein consists in the repetition of one or more amino acids. Contrary to the conjugates described in US 2018/0345249, the linear protein has no free aldehyde function. This can be easily checked with the 3, 5-dinitrosalicylic acid (DNS) method. The method is based on the simultaneous oxidation of aldehydes and the reduction of DNS to 3-amino-5-nitrosalicylicacid upon the application of alkaline conditions and heat, which absorbs light at 540 nm (Sumner, J. B., and Graham, V. A.: Dinitrosalicylic Acid: A Reagent for the Estimation of Sugar in Normal and Diabetic Urine, J. Biol. Chem. 47:5-9 (June) 1921).

In particular, the used linear protein is non-allergenic, which is a known problem for carrier proteins such as BSA and HSA.

According to specific embodiments, the linear protein is made of a repetition of a single amino acid and so, is a homo-poly-amino acid. These are distinguished from common peptides and proteins composed of different kinds of amino acid. Examples are: polylysine, in particular poly-L-lysine, poly-D-lysin and poly-L,D-lysin; poly-lysine-leucine; polyarginine, in particular poly-L-arginine; polyglutamic acid, in particular poly-L-glutamic acid and poly-D-glutamic acid; polyornithine, in particular poly-L-ornithine; polyhomoarginine, in particular poly-L-homoarginine. In particular, before grafting, the linear protein has pendant amino function —NH₂ which are available for the grafting of the oligo or polysaccharide molecules. So, the ungrafted linear protein includes primary amino functions —NH₂ which are pendant from the main chain of the linear protein, so on lateral positions or lateral chains, such as polylysine and polyarginine. In very interesting embodiments, the linear protein is a polylysine, in particular poly-L-lysine.

The carbohydrate nomenclature follows the recommendations by the IUPAC-IUB Commission on Biochemical Nomenclature (CBN for Lipids: Eur. J. Biochem. (1998) 257, 293). In one embodiment, Gal, Glc, GlcNAc, GalNAc and NeuAc are of the D-configuration, Fuc of the L-configuration, and all monosaccharide units are present in the pyranose form, except specified otherwise i.e. when it is mentioned they are open and/or acyclic.

For instance, Fucα2Gal and Fucα1-2Gal designates the same oligosaccharide; GalNAcα3(Fucα2)Galβ3GlcNAcβ3Galβ4Glc and GalNAcα1-3(Fucα1-2)Galβ1-3GlcNAcβ1-3Galβ1-4Glc designates the same oligosaccharide: this oligosaccharide is the blood group A hexaose type 1, named 6A1, A6-1 or A61 in the examples, and is particularly interesting in the invention.

As used herein any monosaccharide written between regular brackets i.e. ( ) is branched from the main saccharidic chain and attached to the next in line monosaccharide of said saccharidic chain as further exemplified below. For example, formula X(Fucα2)Galβ3(Y)TV, can schematically be represented by:

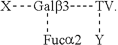

The conjugates of the invention are obtained by grafting, via a covalent bond, n saccharidic chains X(Fucα2)Galβk(Y)TV (with k, X, Y, T and V as defined for formula (II)) on a linear protein W. The attachment is made via the mono-saccharide present at the reducing end of the saccharidic chain (II), namely the end of V when present, otherwise T when present, otherwise Galβk if T and V are absent.

The coupling may be direct: it means that the reaction leading to the grafting involves directly the polysaccharide or oligosaccharide of formula X(Fucα2)Galβk(Y)TV (II), with n, k, X, Y, T and V as defined herein, and the linear protein. According to another possible indirect route, it is possible to use polysaccharides or oligosaccharides of formula X(Fucα2)Galβk(Y)TV previously modified for carrying, on their reducing end, a reactive function which is able to react with the linear protein, and in particular some of its free primary amino functions for instance in the case of polylysine or polyarginine. Some examples of such reactive functions are aldehyde, —NH₂, or ketone which can be introduced by conventional techniques. The reactive function may be linked to the saccharidic chain (II) by a spacer arm, for instance, but not only, polyethylene glycol.

The easiest and more advantageous route of grafting the polysaccharide or oligosaccharide of formula X(Fucα2)Galβk(Y)TV (II) on the linear protein is to covalently couple them on the linear protein, by reductive amination of their reducing end. This route is suitable when the ungrafted linear protein carries pendant primary amino functions —NH₂. This route will be explained in more details: the reducing end of an oligo or polysaccharide is present in an acyclic and several cyclic forms in equilibrium. The acyclic form presents an aldehyde function that makes it reactive with especially toward primary amino functions of the linear protein. This reversible condensation leads to a Schiff base that is stabilized by reduction when it reacts with a reducing agent, for instance borohydride derivatives. So, in the final conjugate, the terminal saccharidic unit of V, when V is present, or of T, when T is present and V is absent, or Gal (corresponding to Galβk in the formula (II)), when both T and V are absent, is open and acyclic and bond to an azote of the linear protein, after elimination of a molecule of H₂O. In that case, the obtained covalent bond between the polysaccharidic or oligosaccharidic part and the linear protein of the conjugate is a secondary amine linkage which is obtained by the reaction of a free amino function of the (ungrafted linear protein) and the aldehyde group of the reducing end of the saccharidic chain corresponding to formula X(Fucα2)Galβk(Y)TV (II), with n, k, X, Y, T and V as defined herein. So, the saccharidic unit by which the covalent bond is formed is denatured: this unit is open and acyclic and a hydrogen atom and an oxygen atom are lost, for obtaining the secondary amine bond. So, in that cases, the saccharidic chains (II) are covalently coupled on the linear protein, by the saccharidic unit corresponding to their reducing end which is open and acyclic and has been coupled by reductive amination, by its aldehyde function to a pending amino function —NH₂ which was present on the linear protein before grafting. So, in that case, before the grafting of the saccharidic chains X(Fucα2)Galβk(Y)TV (II), the linear protein named ungrafted) includes primary amino function —NH₂, on lateral positions or lateral chains, which are available for grafting. After grafting, most of the time, some of these functions will remain present. Of course, it will depend of the density of grafting.

In particular, this method is suitable for the grafting of saccharidic chains (II) on polylysine.

When V is a mono, or an oligosaccharide, according to specific embodiments, its reducing end will be Gal or Glc or any monosaccharide suitable for such reductive amination reactions, such as Man.

In the present invention, it was found that several molecules of the saccharidic chain X(Fucα2)Galβk(Y)TV (II) as defined in the invention, grafted on a specific type of carrier which is a linear protein, were able to decrease the adhesion of F18⁺ E. coli to porcine intestinal villi. In vitro, a reduction of at least 40% of the adhesion was obtained, in comparison with the use of at least 10 times higher concentration of other conjugates including other carriers instead of linear protein, as shown in the examples.

In more specific embodiments, the linear protein W is a polylysine, in particular a poly-L-lysine, such as ε-Poly-L-lysine (s-PL) which is a homo-poly-amino acid characterized by a peptide bond between the carboxyl and ε-amino groups of L-lysine. Polylysine is known for its high thermal stability, is well known as supplement in feed and is considered safe.

It was demonstrated herein that the conjugation of several molecules of the saccharidic chain X(Fucα2)Galβk(Y)TV as defined in the invention, to polylysine, and more generally to a linear protein as defined in the invention, results in a highly effective conjugate that inhibits adhesion of bacteria, in particular to porcine intestinal villi, at very low concentrations.

Other linear protein can be used instead of polylysine, such as poly-L-arginine, poly-L-glutamic acid, poly-L-ornithine, poly-L-homoarginine, poly-D-glutamic acid.

Due to the high efficacy of the conjugates of the invention, only low quantity of the conjugate according to the invention per kg food or per liter of drink is required to be effective, in the feeding of mammals and in particular of piglets. These quantities are particularly suitable when the linear protein W is a polylysine, in particular a poly-L-lysine, such as ε-poly-L-lysine.

According to particular embodiments, the linear protein, and particularly polylysine, has an average molecular weight in weight (Mw) in the range from 2000 to 33000 g/moL, in particular in the range from 3200 to 6850 g/moL. This average molecular weight can be determined by MALDI-TOF mass spectrometry.

According to particular embodiments, the linear protein, and particularly polylysine, has an average degree of polymerization (DP) in the range from 15 to 240, in particular in the range from 20 to 50. This average degree of polymerization DP can be determined by MALDI-TOF mass spectrometry.

In the conjugates (I) of the invention, n, which is the number of saccharidic chains grafted on the linear protein, is 8 or more, in particular is 9 or more, 10 or more, 11 or more, 12 or more, 13 or more, 14 or more, 15 or more, 20 or more or 30 or more. A too high rate could lead to steric hindrance and increase the costs to produce the conjugate. So, according to particular embodiments, n is less than 240, particularly, less than 100, more particularly less than 50, in particular less than 45, in particular less than 40. More specifically, n is in the range from 8 to 240, for instance from 8 to 100, from 8 to 50, from 8 to 40, and in particular in the range from 10 to 35, more specifically in the range from 12 to 35.

In some embodiments, the invention provides conjugates (I) wherein when X is absent and Y is absent, then T is absent or is ZNAcε3, with Z is Glc.

In other embodiments, the conjugates (I) are characterized in that X is Galα3 or GalNAcα3; Z is Glc or Gal and Y is absent.

In another embodiment of the conjugates (I), V is UGalβ4Glc; wherein U is absent, is Galα4, Galβ3GlcNAcβ3, or (Fucα2)Galβ3GlcNAcβ3.

In another embodiment, T is ZNAcε3 and ε is β and/or Z is Glc.

In a further embodiment, X, Y, T and V are absent in the conjugate (I).

In a further aspect, the present invention provides a conjugate with grafted saccharidic chains of formula (IIa): X(Fucα2)Galβk(Y)ZNAcε3UGalβ4Glcβ1 wherein U is absent, is Galα4, Galβ3GlcNAcβ3, or (Fucα2)Galβ3GlcNAcβ3, and X, Y, E, Z and W are as defined for formula (II), k is 4 or preferably 3.

Another interesting group of conjugates are those conjugates with grafted saccharidic chains of formula (II) or (IIa) wherein ε is β.

Yet another interesting group of conjugates are those conjugates with grafted saccharidic chains of formula (II) or (IIa), wherein Z is Glc.

In one embodiment, in the conjugate of the invention, the grafted saccharidic chains X(Fucα2)Galβk(Y)TV (II) are selected or obtained from the group consisting of:
Fucα2Gal;
Fucα2Galβ3GlcNAcβ3Gal;
GalNAcα3(Fucα2)Galβ4Glc (A4-5);
Fucα2Galβ3GlcNAcβ3Galβ4Glc (H5-1);
Galα3(Fucα2)Galβ3GlcNAcβGal;
Galα3(Fucα2)Galβ3GlcNAcβ3Galβ4Glc (B6-1);
GalNAcα3(Fucα2)Gal;
GalNAcα3(Fucα2)Galβ3GlcNAcβ3Gal (A5-1);
GalNAcα3(Fucα2)Galβ3GlcNAcβ3Galβ4Glc (A6-1);
GalNAcα3(Fucα2)Galβ4GlcNAcβ3Galβ4Glc (A6-2);
GalNAcα3(Fucα2)Galβ3(Fucα4)GlcNAcβ3Gal;
GalNAcα3(Fucα2)Galβ3(Fucα4)GlcNAcβ3Galβ4Glc;
Galα3(Fucα2)Galβ3(Fucα4)GlcNAcβ3Gal;
Galα3(Fucα2)Galβ3(Fucα4)GlcNAcβ3Galβ4Glc;
GalNAcα3(Fucα2)Galβ3GlcNAcβ3Galβ3GlcNAcβ3Gal;
GalNAcα3(Fucα2)Galβ3GlcNAcβ3Galβ3GlcNAcβ3Galβ4Glc;
GalNAcα3(Fucα2)Galβ3GalNAcβ3Galα4Gal;
GalNAcα3(Fucα2)Galβ3GalNAcβ3Galα4Galβ4Glc;
GalNAcα3(Fucα2)Galβ3GalNAcα3(Fucα2)Galβ3GlcNAcβ3Gal; and
GalNAcα3(Fucα2)Galβ3GalNAcα3(Fucα2)Galβ3GlcNAc 3Galβ4Glc.

In one embodiment, the conjugate of the invention is a conjugate of or is obtained by grafting n saccharidic chains of formula (II) or (IIa) selected from the group consisting of:
Fucα2Gal;
Fucα2Galβ3GlcNAcβ3Gal;
GalNAcα3(Fucα2)Galβ4Glc; (A4-5)
Fucα2Galβ3GlcNAcβ3Galβ4Glc (H5-1);
Galα3(Fucα2)Galβ3GlcNAcβ3Gal;
Galα3(Fucα2)Galβ3GlcNAcβ3Galβ4Glc (86-1);
GalNAcα3(Fucα2)Gal;
GalNAcα3(Fucα2)Galβ3GlcNAcβ3Gal (A5-1);
GalNAcα3(Fucα2)Galβ3GlcNAcβ3Gal$4Glc (A6-1);
GalNAcα3(Fucα2)Galβ4GlcNAcβ3Galβ4Glc (A6-2);
GalNAcα3(Fucα2)Galβ3(Fucα4)GlcNAcβ3Gal;
GalNAcα3(Fucα2)Galβ3(Fucα4)GlcNAcβ3Galβ4Glc;
Galα3(Fucα2)Galβ3(Fucα4)GlcNAcβ3Gal;
Galα3(Fucα2)Galβ3(Fucα4)GlcNAcβ3Galβ4Glc;
GalNAcα3(Fucα2)Galβ3GlcNAcβ3Galβ3GlcNAcβ3Gal;
GalNAcα3(Fucα2)Galβ3GlcNAcβ3GalβGlcNAcβ3Galβ4Glc;
GalNAcα3(Fucα2)Galβ3GalNAcβ3Galα4Gal;
GalNAcα3(Fucα2)Galβ3GalNAcβ3Galα4Galβ4Glc;
GalNAcα3(Fucα2)Galβ3GalNAcα3(Fucα2)Galβ3GlcNAcβ3Gal; and
GalNAcα3(Fucα2)Galβ3GalNAcα3(Fucα2)Galβ3GlcNAcβ3Galβ4Glc,
on a linear protein as defined herein, in particular on polylysine, more particularly poly-L-lysine; and wherein n is 8 or more, in particular 10 or more, 11 or more, 12 or more, 13 or more, 14 or more, 15 or more, 20 or more or 30 or more. In one embodiment n is less than 45, in particular less than 40. More specifically, n is in the range from 8 to 40, more specifically in the range from 10 to 35, and even more specifically in the range from 12 to 35.

According to a specific embodiment, the grafting of these saccharidic chains is made by reductive amination. In that case, the above mentioned conjugates have the partial following formula (Ip), in the grafting area when W is ε-poly-L-lysine:

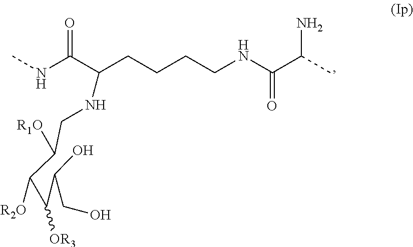

in which, R₁, R₂ and R₃ are as defined in Table A, for each grafted saccharidic chain (II) specifically described herein.

TABLE A

| Saccharidic chain (II) | R1 | R2 | R3 |
|---|---|---|---|
| Fucα2Gal | Fuc | H | H |
| Fucα2Galβ3GlcNAcβ3Gal | H | Fucα2Galβ3GlcNAc | H |
| Fucα2Galβ3GlcNAcβ3Galβ4Glc (H5-1) | H | H | Fucα2Galβ3GlcNAcβ3Gal |
| GalNAcα3(Fucα2)Galβ4Glc (A4-5) | H | H | GalNAcα3(Fucα2)Gal |
| GalNAcα3(Fucα2)Galβ3GlcNAcβ3 | H | GalNAcα3(Fucα2)Galβ3 | H |
| Gal | | GlcNAc | |
| Galα3(Fucα2)Galβ3GlcNAcβ3 | H | H | Galα3(Fucα2)Galβ3GlcNAcβ3Gal |
| Galβ4Glc (B6-1) | | | |
| GalNAcα3(Fucα2)Galβ3GlcNAcβ3 | H | GalNAcα3(Fucα2) | H |
| Gal (A5-1) | | Galβ3GlcNAc | |
| GalNAcα3(Fucα2)Galβ3GlcNAcβ3 | H | H | GalNAcα3(Fucα2)Galβ3GlcNAcβ3Gal |
| Galβ4Glc (A6-1) | | | |
| GalNAcα3(Fucα2)Galβ4GlcNAcβ3 | H | GalNAcα3(Fucα2)Galβ4 | H |
| Galβ4Glc (A6-2) | | GlcNAcβ3Gal | |
| GalNAcα3(Fucα2)Galβ3(Fucα4) | H | GalNAcα3(Fucα2) | H |
| GlcNAcβ3Gal | | Galβ3(Fucα4)GlcNAc | |
| GalNAcα3(Fucα2)Galβ3(Fucα4) | H | H | GalNAcα3(Fucα2)Galβ3(Fucα4) |
| GlcNAcβ3Galβ4Glc | | | GlcNAcβ3Gal |
| Galα3(Fucα2)Galβ3(Fucα4)GlcNAcβ3 | H | Galα3(Fucα2)Galβ3 | H |
| Gal | | (Fucα4)GlcNAc | |
| Galα3(Fucα2)Galβ3(Fucα4)GlcNAcβ3 | H | H | Galα3(Fucα2)Galβ3(Fucα4) |
| Galβ4Glc | | | GlcNAcβ3Gal |
| GalNAcα3(Fucα2)Galβ3GlcNAcβ3 | H | GalNAcα3(Fucα2)Galβ3 | H |
| Galβ3GlcNAcβ3Gal | | GlcNAcβ3Galβ3GlcNAc | |
| GalNAcα3(Fucα2)Galβ3GlcNAcβ3 | H | H | GalNAcα3(Fucα2)Galβ3GlcNAcβ3 |
| Galβ3GlcNAcβ3Galβ4Glc | | | Galβ3GlcNAcβ3Gal |
| GalNAcα3(Fucα2)Galβ3GalNAcβ3 | | Galα4Gal | H |
| Galα4Gal | | | |
| GalNAcα3(Fucα2)Galβ3GalNAcβ3 | H | H | GalNAcα3(Fucα2)Galβ3GalNAcβ3 |
| Galα4Galβ4Glc | | | Galα4Gal |
| GalNAcα3(Fucα2)Galβ3GalNAcα3 | H | GalNAcα3(Fucα2)Galβ3 | H |
| (Fucα2)Galβ3GlcNAcβ3Gal | | GalNAcα3(Fucα2)Galβ3 | |
| | | GlcNAc | |
| GalNAcα3(Fucα2)Galβ3GalNAcα3 | H | H | GalNAcα3(Fucα2)Galβ3GalNAcα3 |
| (Fucα2)Galβ3GlcNAcβ3Galβ4Glc | | | (Fucα2)Galβ3GlcNAcβ3Gal |

In a specific embodiment, the invention provides a conjugate selected from the group consisting of:
[GalNAcα3(Fucα-2)Galβ4Glc′]ₙ-W (A4-5) with n=17 or 30, or more generally n being in the range 12 to 35, and W is ε-poly-L-lysine wherein the covalent bond between a L-lysine unit and Glc which is open and acyclic (Glc′) is:

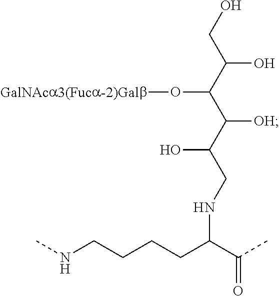

[GalNAcα3(Fucα2)Galβ3GlcNAcβ3Gal′]ₙ-W (A5-1) with n=21, or more generally n being in the range 12 to 35, and W is ε-poly-L-lysine wherein the covalent bond between a L-lysine unit and Gal which is open and acyclic (Gal′) is:

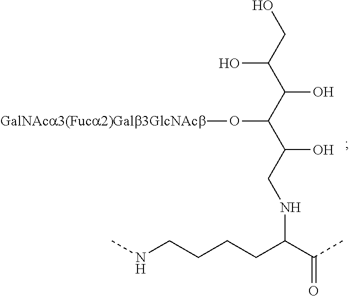

[GalNAcα3(Fucα2)Galβ3GlcNAcβ3Galβ4Glc′]ₙ-W (A6-1) with n=14, 15, 17, 19 or 31, or more generally n being in the range 12 to 35, and W is ε-poly-L-lysine wherein the covalent bond between a L-lysine unit and Glc which is open and acyclic (Glc′) is:

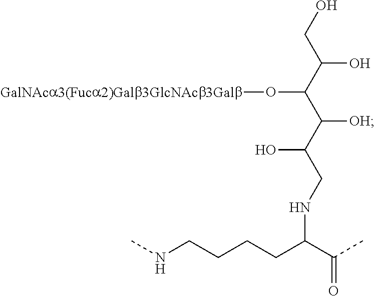

[Galα3(Fucα2)Galβ3GlcNAcβ3Galβ4Glc′]ₙ-W (B6-1) with n being in the range 12 to 35, and W is ε-poly-L-lysine wherein the covalent bond between a L-lysine unit and Glc which is open and acyclic (Glc′) is:

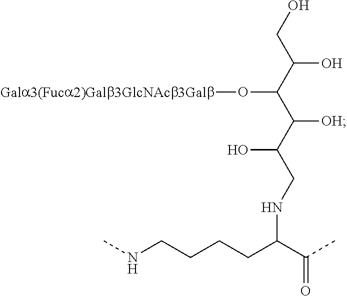

In other words, the invention provides a conjugate selected from the group consisting of:
the conjugate obtained by grafting n GalNAcα3(Fucα2)Galβ4Glc (A4-5) with n=17 or 30, or more generally n being in the range 12 to 35, on ε-poly-L-lysine, by reductive amination;
the conjugate obtained by grafting n GalNAcα3(Fucα2)Galβ3GlcNAcβ3Gal (A5-1) with n=21, or more generally n being in the range 12 to 35, on ε-poly-L-lysine, by reductive amination;
the conjugate obtained by grafting n GalNAcα3(Fucα2)Galβ3GlcNAcβ3Galβ4Glc (A6-1) with n=14, 15, 17, 19, 31, or more generally n being in the range 12 to 35, on ε-poly-L-lysine, by reductive amination;
the conjugate obtained by grafting n Galα3(Fucα2)Galβ3GlcNAcβ3Galβ4Glc (B6-1) with n being in the range 12 to 35, on ε-poly-L-lysine, by reductive amination.

These specific conjugates have a very high efficacy, in particular in the feed of mammals, and specifically of piglets.

According to particular embodiments, the conjugates (I) of the invention and in particular those previously mentioned have a weight average molecular weight Mw in the range from 5000 to 410000 g/moL, in particular in the range from 5600 to 86000 g/moL. It can be measured by 1H-NMR.

The average molecular weight of a conjugate according to the invention is calculated on the basis of the weight average molecular weight Mw of the linear protein (measured by mass spectrometry MALDI-TOF) and the average number of saccharidic chains grafted on one molecule of linear protein (determined by 1H-NMR on the basis of the ratio between the proton signals of the saccharidic chain and the proton signals of the linear protein). The formula of determination of the mean molecular weight is: linear protein average molecular weight+(saccharidic chain average molecular weight−17)×average number of saccharidic chains grafted on one linear protein. In this formula, 17 corresponds to the loss of 1 oxygen (16) from de monosaccharide of the reducing end of the sugar and the loss of 1H from the free NH₂ function carried by polylysine.

Production of the Conjugates

Many suitable linear proteins are commercially available.

The polysaccharides or oligosaccharides X(Fucα2)Galβk(Y)TV, where k, X, Y, T and V are as defined in formula (II) or (IIa) are described in WO 2010/037785 or US 2018/0345249. These oligosaccharides or polysaccharides are produced by conventional techniques. In particular, most of the oligosaccharides may be obtained from fermentation of engineered E. coli and can be purified by any suitable techniques, like HPLC-reverse phase. The technology of production of the oligosaccharides is described in WO 2001/004341A1 (Method for producing oligopolysaccharides). They are available through the commercial offer of Elicityl (Crolles, France).

The saccharidic chains X(Fucα2)Galβk(Y)TV, where k, X, Y, T and V are as defined in formula (II) or (IIa), are grafted on the linear protein, according to conventional techniques. The number of grafted saccharidic chain X(Fucα2)Galβk(Y)TV may be modulated, by the initial concentration of the polysaccharide or oligosaccharide X(Fucα2)Galβk(Y)TV (II) potentially functionalized in the reaction mixture.

In particular, the polysaccharide or oligosaccharide is grafted on the linear protein, for instance polylysine, in particular poly-L-lysine, by reductive amination. In that case the reaction of grafting involves directly the polysaccharide or oligosaccharide of formula X(Fucα2)Galβk(Y)TV where k, X, Y, T and V are as defined in formula (II) or (IIa) and the (ungrafted) linear protein, which carries pendant —NH₂ functions. They react together under reductive conditions, in particular in the presence of a reductive agent. The reaction between primary amino groups of the polylysine, and the reducing-end aldehyde group of the polysaccharide or oligosaccharide of formula X(Fucα2)Galβk(Y)TV leads to the formation of an imine bond, reduced by a reducing agent such as NaBH₃CN, NaBH₄, picoline borane or H2 with appropriate catalyst to obtain a stable secondary amine link. The monosaccharide at the reducing end of the oligosaccharide is opened during the reductive amination reaction.

Classically, such reductive amination reaction can be carried out in water, or in an aqueous solution, such as borate sodium buffer or phosphate buffer, at pH between 7.5 to 9.5. The molar ratio of the polysaccharide or oligosaccharide X(Fucα2)Galβk(Y)TV, where k, X, Y, T and V are as defined in formula (II) or (IIa), on the linear protein, may be in the range from 2 to 900, in particular in the range from 4 to 450. The molar ratio of the polysaccharide or oligosaccharide of formula X(Fucα2)Galβk(Y)TV (II), where k, X, Y, T and V are as defined herein for formula (II) or (IIa), on the reducing agent, may be in the range from 9 to 18000, in particular in the range from 18 to 9000. Typically, the reaction will be carried out, by maintaining the mixture of the reactive components, namely, the polysaccharide or oligosaccharide of formula X(Fucα2)Galβk(Y)TV (II), where k, X, Y, T and V are as defined herein for formula (II) or (IIa), the linear protein and the reducing agent, at a temperature in the range from 15 to 60° C., in particular from 40 to 50° C., during 1 to 7 days, in particular from 4 to 5 days. The obtained conjugates are, conventionally, purified by dialysis, gel permeation or ultrafiltration and analysed using MALDI-TOF MS and ¹H-NMR spectroscopy.

Another way of grafting involves the (ungrafted) linear protein and the polysaccharides or oligosaccharides X(Fucα2)Galβk(Y)TV, where k, X, Y, T and V are as defined herein for formula (II) or (IIa), which have been previously functionalized, in order to allow their grafting on the linear protein. This route is, for instance, suitable when the linear protein do not carry lateral —NH₂ functions available for grafting. Any conventional technique of conjugation, for instance the isocyanate route may be used. It is also possible to introduce an amine function —NH₂ at the reducing end of polysaccharide or oligosaccharide X(Fucα2)Galβk(Y)TV (II), for obtaining a functionalized polysaccharide or oligosaccharide X(Fucα2)Galβk(Y)TV. This amine function is able to react with —COOH carried by linear protein, such as polyglutamic acid, and lead to the formation of an amide bond. The formation of amide bonds as described before can be carried out according to any procedure known to the person skilled in the art. A common method comprises the activation of the carboxylic acid with a carbodiimide, thus facilitating the coupling to an amine. Other functionalization, such as aldehyde or ketone, are also possible and may be introduced directly or by any suitable spacer arm.

Composition

In a further embodiment, the present invention provides a composition comprising one or more of the conjugates according to the invention as defined herein, and a diluent or excipient. Mixtures of different types of conjugates can also be used. Possible diluents are water or ethanol. Possible excipients include, but are not limited to, saline, buffered saline, dextrose, water, glycerol, ethanol, and combinations thereof.

It is of particular advantage to use a conjugate according to the invention as part of a nutritional composition (food or drink) including food- and feedstuff. In a particular embodiment, the composition is a feed additive composition. A “feed” and a “food”, respectively, means any natural or artificial diet, meal or the like or components of such meals intended or suitable for being eaten, taken in, digested, by a non-human animal and a human being, respectively. As used herein, the term “food” is used in a broad sense and covers food and food products for humans as well as food for non-human animals (i.e. a feed). The term “feed” is used with reference to products that are fed to animals in the rearing of livestock. The terms “feed” and “animal feed” are used interchangeably. Hence a conjugate according to the invention can be comprised in animal solid and liquid feeds and drinking water of animals.

One embodiment of the invention is pig feed supplemented with one or more of the conjugates (I) specified herein. Suitable pig feed includes, but is not limited to, starter feed, weaning feed or fattening feed.

The composition described herein may be presented in various physical forms. The conjugate according to the invention provided herein may optionally be admixed with a dry formulation of additives including, but not limited to, growth substrates, enzymes, sugars, carbohydrates, extracts and growth promoting micro-ingredients.

Uses for Nutritional Uses

The invention also concerns the conjugates as defined in the invention, for their use in the treatment of an F18+ E. coli infection in mammals, and in particular pigs. In particular, in this use, the conjugate binds to F18+ E. coli, F18 fimbriae, F18 adhesin, FedF or to the receptor binding domain of FedF.

Post-weaning diarrhoea is a multifactorial disease complex in which piglets develop diarrhoea as a result of stress, change in food, ETEC and other pathogens such as rotavirus. However, it is commonly accepted that E. coli (ETEC and/or VTEC) are the most important pathogens of this disease complex. In particular, the conjugates of the invention are effective against pathogenic F18+ E. coli producing enterotoxins such as e.g. LT, ST, and SLTx toxins.

So, the conjugates of the invention are very useful, for the treatment post weaning diarrhea and edema disease, for pigs. In general, the conjugates and compositions of the present invention may be employed for preventive or prophylaxis treatments of intestinal infections, in particular gastrointestinal infections, more in particular for effective inhibition of pathogens, especially adhesion of diarrhea causing F18+ Escherichia coli bacteria. F18 fimbriae are thin, flexible polymeric filaments used by F18⁺ E. coli strains to attach to host tissues. With the term “F18 adhesin” is meant a fimbrial lectin that mediates adhesion to the F18R. “FedF” refers to the minor adhesive subunit of F18 fimbriae that is expressed by the fed gene cluster. With the term “receptor binding domain of FedF” is meant a region located at the amino-terminal half of the FedF protein essential for binding and mapped between amino acid 60 and 109. As referred to herein, “F18 receptor or F18R” is a specific carbohydrate receptor on the porcine intestinal epithelium to which F18⁺ E. coli can attach.

In one embodiment, the invention provides the conjugates (I) and compositions specified herein, in particular the specific conjugates described herein for use as a medicament.

In another embodiment, the present invention relates to the use of conjugates (I) and compositions specified herein, in particular the specific conjugates described herein, for the manufacture of a medicament for preventing, inhibiting or treating of F18⁺ E. coli infections in mammals, in particular pigs, such as piglets, growing pigs and sows.

The invention furthermore relates to a conjugate (I) according to the invention, in particular the specific conjugates described herein, for use in preventing, inhibiting or treating of F18⁺ E. coli infections in mammals such as pigs (in particular, piglets, growing pigs and sows), in particular in preventing, inhibiting or treating of diarrhea, more in particular post weaning diarrhea and edema disease.

With the terms “preventing”, “inhibiting” or “treating” is meant any treatment of a disease and/or condition in a mammal, particularly an animal, and includes: (i) preventing a disease and/or condition from occurring in a mammal which may be predisposed to the disease and/or condition but has not yet been diagnosed as having it; (ii) inhibiting the disease and/or condition, i.e., arresting its development; (iii) relieving the disease and/or condition, i.e., causing regression of the disease and/or condition.

Thus, in a further aspect, the present invention provides a method for preventing, treating or ameliorating a medical condition related to F18⁺ E. coli infections which comprises administering to a mammalian subject a (therapeutically) effective amount of a conjugate according to the invention, optionally in combination with a further excipient or diluent, in an amount effective to reduce F18⁺ E. coli infections. Conjugates of the present invention may be employed alone or in conjunction with other compounds, such as therapeutic compounds.

The conjugate or composition will be adapted to the route of administration, for instance an oral route. Administration of the conjugates of the invention may be in the form of pills, tablets, capsules, powders, solutions, suspensions, pastes, gels, and the like.

The conjugate or composition or formulation to be administered will, in any event, contain a quantity of the active conjugate, and eventually additional active compound(s), in an amount effective to alleviate the symptoms of the animal being treated.

The terms “animal” and “subject” are used interchangeably herein. An animal includes mammals, in particular all non-ruminant (including humans) and ruminant animals. In a particular embodiment, the animal is a mono-gastric animal, more in particular pigs and swine, such as piglets, growing pigs and sows.

The exact dosage and frequency of administration of the conjugates and compositions according to the invention depends on the particular conjugate used, the particular condition being treated, the severity of the condition being treated, the age, weight, gender, diet, time of administration and general physical condition of the particular subject, the mode of administration as well as other medication the subject may be taking, as is well known to those skilled in the art. Furthermore, it is evident that the effective daily amount may be lowered or increased depending on the response of the treated subject and/or depending on the evaluation of the veterinarian prescribing the conjugates of the instant invention.

According to interesting embodiments, the inclusion rate of the conjugate in the feed is at least 0.0005% or 5 mg/kg and/or up to 0.005% or 50 mg/kg, in particular at least 0.0008% or 8 mg/kg and/or up to 0.002% or 20 mg/kg.

According to advantageous embodiments, the concentration of the conjugate in the drink, in particular drinking water is at least 0.05% or 0.05 g/100 L and/or up to 5% or 5 g/100 L, in particular at least 0.1% or 0.1 g/100 L and/or up to 2% or 2 g/100 L.

Nevertheless, dosage forms or compositions containing a conjugate according to the invention in the range of 0.05 to 100% may be prepared. Depending on the mode of administration, the composition will preferably comprise from 0.05 to 99% by weight, more preferably from 0.1 to 70% by weight of a conjugate according to the invention, all percentages being based on the total composition.

Formulation of Therapeutic Composition

For oral administration, a pharmaceutically acceptable non-toxic composition is formed by the incorporation of any of the normally employed excipients (i.e. pharmaceutically acceptable carriers), such as, for example, pharmaceutical grades of mannitol, lactose, cellulose, cellulose derivatives, sodium crosscarmellose, starch, magnesium stearate, sodium saccharin, talcum, glucose, sucrose, magnesium, carbonate, and the like. Such compositions take the form of solutions, suspensions, tablets, pills, capsules, powders, sustained release formulations and the like.

Solid forms can be suitable for solution or suspension in liquid or as emulsions. Suitable excipients are, for example, water, saline, dextrose, glycerol, ethanol or the like. In addition, if desired, the pharmaceutical compositions, food or drink additive to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

The percentage of the conjugate of the present invention contained in a specific formulation such as e.g. a solution or suspension is highly dependent on the specific nature thereof, as well as the activity of the conjugate and the needs of the subject. A therapeutically effective amount (i.e. an amount effective to have a therapeutically beneficial effect, in particular to modulate a F 18⁺ E. coli infection) is used.

Packs and Kits

Finally, the invention further relates to packs including pharmaceutical packs and kits comprising one or more containers filled with one or more of the ingredients of the aforementioned conjugates, compositions, food or drink additives and pig feed of the invention or methods of the invention.

This invention provides for an article of manufacture comprising a packaging and a pharmaceutical agent, wherein (a) the pharmaceutical agent is one of the conjugates according to the invention, and (b) the packaging comprises a label indicating the use of the pharmaceutical agent for treating a subject, in particular as a medicine or for the treatment of F18⁺ E. coli infections in pigs, such as piglets, growing pigs and sows.

This invention will be better understood by reference to the Experimental details that follow, but those skilled in the art will readily appreciate that these are only illustrative of the invention as described more fully in the description and the claims. Additionally, throughout this application, various publications are cited. The disclosure of these publications is hereby incorporated by reference into this application to describe more fully the state of the art to which this invention pertains.

### Examples

### Materials and Methods

### 1^{st} Part—In Vitro Testing

In Vitro Villous Adhesion Assay

The nature of the F18R on porcine small intestinal villous enterocytes was investigated using an in vitro villous adhesion inhibition assay (Cox et al., 1993). Briefly, a 20 cm intestinal segment was collected from the mid jejunum of a euthanized pig, rinsed three times with ice cold PBS, and fixed with Krebs-Henseleit buffer (160 mM, pH 7.4) containing 1% (v/v) formaldehyde for 30 min at 4° C. Thereafter, the villi were gently scraped from the mucosae with a glass slide and stored in Krebs-Henseleit buffer at 4° C. Treatment of villi with acetone, methanol, 1% Triton® X-100, 10 mM NaIO₄ in 0.2M sodium acetate, pH 4.5, or 0.2M sodium acetate, pH 4.5, without NaIO₄, respectively, was performed at room temperature on a rotating wheel in a volume of 500 μl during 1 h. Next, the villi were washed 6 times with Krebs-Henseleit buffer followed by addition of 4×10⁸ bacteria of the F18 positive (F18+) reference E. coli strain (107/86), or the F4ac-expressing E. coli strain GIS 26 (a field isolate from a faecal sample of a pig, Van den Broeck et al., 1999), to an average of 50 villi in a total volume of 500 μl PBS, supplemented with 1% (w/v) D-mannose in order to prevent adhesion mediated by type 1 pili. These mixtures were incubated at room temperature for 1 h while being gently shaken. Villi were examined by phase-contrast microscopy at a magnification of 600, and the number of bacteria adhering along 50 μm brush border was quantitatively evaluated by counting the number of adhering bacteria at 20 randomly selected places, after which the mean bacterial adhesion was calculated. For each test villi of at least two different F18R+ pigs were used and this with minimal 3 repeats.

Bacterial Strains, Culture and Labelling.

The verotoxigenic F18 positive E. coli reference strain 107/86 (serotype 0139:K12:H1, F18ab+, SLT-IIv+) (Bertschinger et al., 1990), and the enterotoxigenic F4ac positive E. coli reference strain GIS 26 (serotype 0149:K91:F4ac, LT+, STa+, STb+), were cultured on BHI agar plates (Oxoid, Basingstoke, Hampshire, England) at 37° C. for 18 h. Subsequently, the bacteria were harvested by centrifugation and resuspended in phosphate-buffered saline (PBS, pH 7.3). The concentration of bacteria in the suspension was determined by measuring the optical density (OD) at 660 nm (OD660). An OD of 1 equals 10⁹ bacteria per ml, as determined by counting colony forming units.

Recombinant E. coli strains expressing whole F18 fimbriae (HB101(pIH120), or F18 fimbriae with deletion of the FedF adhesive subunit (HB101(pIH126)) (Imberechts et al., 1992; 1996), were grown on Iso Sensitest agar plates (Oxoid, Basingstoke, Hampshire, England) supplemented with ampicillin (100 μg/ml) at 37° C. over night. For metabolic labeling, the culture plates were supplemented with 10 μl ³⁵S-methionine (400 μCi; Amersham Pharmacia Biotech). Bacteria were harvested, washed three times in PBS, and resuspended in PBS containing 2% (w/v) bovine serum albumin, 0.1% (w/v) NaN₃ and 0.1% (w/v) Tween® 20 (BSA/PBS/Tween® 20) to a bacterial density of 1×10⁸ colony forming units/ml. The specific activity of bacterial suspensions was approximately 1 cpm per 100 bacteria.

The deletion mutant GIS 26 F4+ STa− STb− LT−, does not produce toxins. It is an isogenic deletion mutant of GIS 26 and generated at the UGent using generated using the bacteriophage lambda recombinase system (Loos et al., 2012). The strain was used as a toxin negative control in the small intestinal segment perfusion test. The F18ac+ E. coli STa⁺ STb⁺ strain 2134P (Tiels et al., 2005) was also used in the intestinal segment perfusion test. Both strains were cultured and harvested as described for GIS 26.

Production of the Oligosaccharides and of the Conjugates

Oligosaccharides were obtained from fermentation of engineered E. coli and purified by HPLC-reverse phase. The method of production of the used oligosaccharides is described in WO2001/004341A1 “Method for producing oligopolysaccharides” and are available through the commercial offer of Elicityl (Crolles, France).

They were grafted on different soluble carriers among which pectin polysaccharide (P), alginate, methacrylate, carboxymethylcellulose of low viscosity (CMCL), carboxymethylcellulose of medium viscosity (CMCM), human serum albumin (HSA), bovine serum albumin (BSA) and Epsilon-poly-L-lysine (PL).

The Epsilon-poly-L-lysine was characterized by an average molecular weight Mw of 4700 g/mol determined by mass spectrometry MALDI-TOF and an average degree of polymerization of 37.

Oligosaccharides grafted on a carrier were among others blood group A type 1 (blood group A hexaose type 1 corresponding to GalNAcα1-3(Fucα1-2)Galβ1-3GlcNAcβ1-3Galβ1-4Glc, named 6A1, A6-1, or A61 or simply A6), blood group H pentaose type 1 (named H5-1 or H51, corresponding to Fucα1-2Galβ1-3GlcNAcβ1-3Galβ1-4Glc), blood group A antigen tetraose type 5 (named A4-5 or A45 corresponding to GalNAcα1-3(Fucα1-2)Galβ1-3Glc) and LNT (Lacto-N-tetraose corresponding to Galβ1-3GlcNAcβ1-3Galβ1-4Glc).

During the fermentation process of blood group A type 1 also blood group H pentaose type 1 (H5-1) and LNT=Lacto-N-tetraose are formed. Therefore, also a less pure A6-1 was used for grafting on PL, a mixture of oligosaccharides containing 85% by weight of A6-1 and 10% by weight of H51+LNT. All the prepared conjugates are summarized in the Table 1 hereafter, which gives several characteristics of the conjugates, carriers (linear protein according to the invention or others) and oligosaccharides.

TABLE 1

| | | | | | Carrier | |
|---|---|---|---|---|---|---|
| | | | | | average | Mean |
| | | | Oligosaccharide | | molecular | rate of |
| | | | molecular | | weight | conjugation |
| Short name | Name | Oligosaccharide | weight | Carrier | Mw | (n) |
| A6-1-P | A6-P | A6-1 | 1056.96 | P | | |
| A6-1-PL 14 | A6-1-PL | A6-1 | 1056.96 | PL | 4700 | 14 |
| | (Coligo) | | | | | |
| A6-1-PL 19 | A6-1-PL | A6-1 | 1056.96 | PL | 4700 | 19 |
| | (Coligo) | | | | | |
| A6-1-PL 15 | A6-1-PL | A6-1 | 1056.96 | PL | 4700 | 15 |
| | (Coligo) | | | | | |
| A6-1-PL 7 | A6-1-PL | A6-1 | 1056.96 | PL | 4700 | 7 |
| A6-CMCL | A6-1-CMC | A6-1-SP1-NH2 | 1354.32 | CMCL | | 0.3 |
| type 1 | Low | | | | | |
| | viscosity | | | | | |
| A6-CMCM | A6-1-CMC | A6-1-SP1-NH2 | 1354.32 | CMCM | | 1 |
| type 1 | Medium | | | | | |
| | viscosity | | | | | |
| A6-CMCL | A6-1-CMC | A6-1-SP1-NH2 | 1354.32 | CMCL | | 3 |
| type 2 | Low | | | | | |
| | viscosity | | | | | |
| A6-CMCM | A6-1-CMC | A6-1-SP1-NH2 | 1354.32 | CMCM | | 9 |
| type 2 | Medium | | | | | |
| | viscosity | | | | | |
| A6-1-PL 17 | A6-1-PL | A6-1 | 1056.96 | PL | 4700 | 17 |
| | (Coligo) | | | | | |
| A6-1-PL 31 | A6-1-PL | A6-1 | 1056.96 | PL | 4700 | 31 |
| | (Variant 1) | | | | | |
| A6-1-PL 6 | A6-1-PL | A6-1 | 1056.96 | PL | 4700 | 6 |
| | (Variant 2) | | | | | |
| A6-1-BSA 6 | A6-1-BSA | A6-1 | 1056.96 | BSA | 66500 | 6 |
| | (Variant 1) | | | | | |
| A6-1-BSA 20 | A6-1-BSA | A6-1 | 1056.96 | BSA | 66500 | 20 |
| | (Variant 2) | | | | | |
| A6-2-BSA 24 | A6-2-BSA | A6-2 | 1056.96 | BSA | 66500 | 24 |
| A6-1-HSA 15 | A6-1-HSA | A6-1 | 1056.96 | HSA | 66500 | 15 |
| A5-1-PL 21 | A5-1-PL | A5-1 | 894.82 | PL | 4700 | 21 |
| A4-5-PL 30 | A4-5-PL | A4-5 | 691.63 | PL | 4700 | 30 |
| | (Variant 1) | | | | | |
| A4-5-PL 17 | A4-5-PL | A4-5 | 691.63 | PL | 4700 | 17 |
| | (Variant 2) | | | | | |
| A6-1/H5-1/LNT- | Mixt-PL | A6-1/H5-1/LNT | | PL | 4700 | 31 |
| PL 31 | (Variant 1) | | | | | |
| A6-1/H5-1/LNT- | Mixt-PL | A6-1/H5-1/LNT | | PL | 4700 | 16 |
| PL 16 | (Variant 2) | | | | | |
| A6-1 | Control 1 | A6-1 | 1056.96 | na | na | na |
| H5-1-PL27 | Control 2 | H5-1 | 853.77 | PL | 4700 | 27 |
| LNT-PL 26 | Control 3 | LNT | 707.63 | PL | 4700 | 26 |
| A6-1/H5-1/LNT | Control 4 | A6-1/H5-1/LNT | | na | na | na |
| PL | Control 5 | na | na | PL | 4700 | na |
| BSA | Control 6 | na | na | BSA | 66463 | na |
| A6-1-FITC | A6-1-FITC | A6-1 | 1056.96 | FITC | | na |

A6-1: Blood group A antigen hexaose type 1 (struture: GalNAcα1-3(Fucα1-2)Galβ1-3GlcNAcβ1-3Galβ1-4Glc; Elicityl commercial reference: GLY037-1)
A61-SP1-NH2: Blood group A antigen hexaose type 1 linked to a spacer (structure: GalNAcα1-3(Fucα1-2)Galβ1-3GlcNAcβ1-3Galβ1-4Glc-NAc-CH2-(1.4-Tz)-(CH2)2-EG3-NH2; Elicityl commercial reference GLY037-1-NAc-sp1-NH2)
A6-2: Blood group A antigen hexaose type 2 (structure: GalNAcα1-3(Fucα1-2)Galβ1-4GlcNAcβ1-3Galβ1-4Glc; Elicityl commercial reference GLY037-2)
A5-1: Blood group A antigen pentaose type 1 (structure: GalNAcα1-3(Fucα1-2)Galβ1-3GlcNAcβ1-3Gal; Elicityl commercial reference GLY036-1)
A4-5: Blood group A antigen tetraose type 5 (structure: GalNAcα1-3(Fucα-2)Galβ1-4Glc; Elicityl commercial reference GLY035-3)
H5-1: Blood group H antigen pentaose type 1 (structure: Fucα1-2Galβ1-3GlcNAcβ1-3Galβ1-4Glc; Elicityl commercial reference GLY033-1)
LNT: Lacto-N-tetraose (structure: Galβ1-3GlcNAcβ1-3Galβ1-4Glc; Elicityl commercial reference GLY010)
PL: ε-polylysine
P: Pectin polysaccharide
CMCL: Carboxymethylcellulose Low viscosity
CMCM: Carboxymethylcellulose medium viscosity
BSA: Bovine serum albumin
FITC: fluorescein isothiocyanate
Mixt of A6-1/H5-1/LNT (A6-1: 85%, H5-1 + LNT: 10%)

The given number of molecules of oligosaccharide/molecule of carrier (n in the case of the conjugate of the invention) was determined by ¹H-NMR on the basis of the ratio between the proton signals of the saccharidic chain and the proton signals of the linear polymer.

In the case of A6-1-PL, the average number n of saccharidic chains grafted on one linear protein is calculated by comparing the peak integrations of 1H NMR spectra. 1H NMR (D20): δ 5.29 (d, H-1 of Fucose), δ 5.21 (d, H-1 of GalNac), δ 4.74 (d, H-1 of GlcNac), δ 4.66 (d, H-1 of Gal), δ 4.50 (d, H-1 of Gal), δ 2.07-2.09 (2 s, COCH3 of GalNac & GlcNac, 6H), δ 1.28 (d, CH3 of fucose, 3H) 1.68 (2 m, 3 protons of PL, 2H), 1.57 (2 m, δ protons of PL, 2H), 1.35 (2 m, γ protons of PL, 2H). Number of protons in A6-1: 14 H; Number of proton in ε-Polylysine: 6H;

$$Ratio = \frac{\frac{\sum proton integral of A 6 - 1}{14}}{\frac{\sum Proton integral of PL}{6}}$$

The formula of determination of the mean molecular weight is: linear protein average molecular weight+(saccharidic chain average molecular weight−17)×average number of saccharidic chains grafted on one linear protein. Positive control was the monomer A6-1 or another monomeric oligosaccharide (A6-2, H5-1 and LNT), depending on the grafted oligosaccharide. In case of mixtures, only the number of grafted A6-1 was determined and mentioned in the Table 1.

The coupling between the oligosaccharides and PL were carried out by reactive amination. The reaction between the primary amino groups of poly-L-lysine and the aldehyde group of the open reducing-end of the oligosaccharide were catalysed by NaBH₃CN (40 M) in borate buffer (0.3 M). The rate of grafting is controlled by the concentration of oligosaccharide (5 to 40 M). The conjugates were purified by dialysis, and the rate of conjugation were analysed using MALDI-TOF MS and 1H-NMR spectroscopy. In particular, conjugates were synthesized by a reductive amination reaction of epsylon-PL (Epsiliseen-H, 4.7 kDa, Siveele, Netherlands) with A6-1 (0.33 molar excess to number of NH₂ on protein, FW: 1056.96, >90%, ref GLY037-1, Elicityl, France). Borate buffer (300 mM, pH 8.5) was used as solvent for the reaction. The reaction proceeded in two steps: first, the formation of a Schiff base between the terminal oligosaccharide aldehyde group and primary amine groups of epsylon-PL, and second, the reduction of the unstable Schiff base to secondary amines. epsylon-PL was dissolved in borate buffer solution at 40° C., and oligosaccharides was subsequently added to the solution, and the mixture was incubated with stirring two hours for the formation of the Schiff base. Sodium cyanoborohydride (NaBH3CN) was then added to the mixture (5 molar excess to oligosaccharide was used) and it was kept under stirring for four more days. Varying the ratio of A6-1/Lys (0.15 to 2) allowed us to control the degree of grafting of oligosaccharide chains onto the epsylon-PL backbone. The resulting conjugates were isolated by dialysis (Pall centramate cassette, 10000 MWCO, PALL Ultrafiltration unit) against 20×volume of pure water to remove the unreacted starting materials.

For HSA, the grafting was carried out on HSA lysine residues via a spacer using the isothiocyanate coupling method. The spacer used was acetyl-phenylenediamine (APD). It was attached to the oligosaccharide by reductive amination. The reducing monosaccharide unit is thus transformed to an aminoalditol. The same route was used for BSA.

As used herein “Coligo” or “Coligo active substance” or “Coligo A6PL” is a glucoconjugate of molecules of A6 grafted on epsilon-polylysin. For Coligo A6PL, the mean rate of conjugation is 14 to 19 moles of oligosaccharide for 1 mole of polylysin. The oligosaccharide part represents 75% to 80% of the weight of Coligo. This type of conjugate has the following formula, at a point of grafting of the A6 oligosaccharide, on a lysine unit:

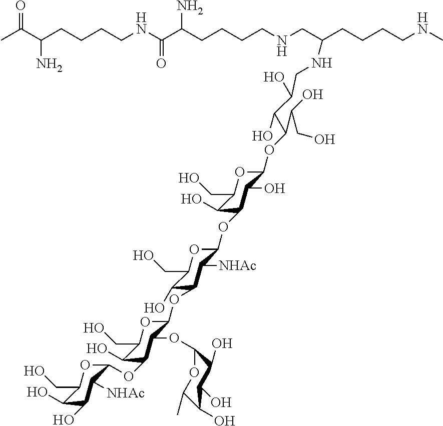

To determine if increasing the conjugation rate could improve the obtained inhibition, two different conjugation rates were produced for A6-1 on CMCL, A6-1 on CMCM and A6-1 on PL as presented in Table 1.

Also different conjugation rates for A6-1 and H5-1 on human serum albumin, for A6-1 on bovine serum albumin and for A6-1, H5-1 and A4-5 on polylysine were produced to test their effect on inhibition of binding of F18+E. coli to villi.

Inhibition of Binding of F18+E. coli by the Conjugates

F18+E. coli (strain 107/86, 8×107 bacteria) were pre-incubated with different concentrations of the conjugates in a final volume of 100 μl followed by incubation with villi isolated from the porcine gut. In parallel, in each assay the blood group A antigen hexaose type 1 (A6-1; 10 mg/ml) and PBS were used as positive and negative inhibition controls, respectively. In some tests additional monomeric oligosaccharides were tested as controls such as the sugar Lacto-N-tetraose (LNT)(negative control), and the blood group H pentaose type 1 (H5-1) (positive control) and B6-1 (positive control). Furthermore, the non-grafted carriers were also tested: pectin, CMCL, CMCM, HSA, BSA, PL. The tested conjugates and/or carriers are as provided in different tested samples are listed in the Table 1.

These compounds were incubated at room temperature for 1 h while being gently shaken. Villi were examined by phase-contrast microscopy and the number of bacteria adhering along 50 μm brush border were quantitatively evaluated by counting the number of adhering bacteria at 20 randomly selected places. Inhibition of adherence is determined by counting the number of adhering bacteria per 250 μm villus. Villi of at least two pigs were used and each test was repeated three times on villi of the same pig to evaluate the inhibition efficacy.

Percentage of inhibition is calculated based on the following formula:

$$( 1 - \frac{\begin{matrix}number of bacteria adhering in \\ the presence of the tested sample\end{matrix}}{\begin{matrix}number of bacteria adhering \\ of control with PBS\end{matrix}} ) \times 100$$

Results were expressed as the mean relative percent of inhibition. This was calculated as:

(the inhibition of adhesion by the tested sample/the inhibition of adhesion by 10 mg/ml A6-1 in the same test)×100.

As such variation between tests was taken into account.

Blocking Assay and Washing Step for Identifying Carrier Based (Non-A6) Inhibition the Binding of Bacteria to the Villi by the Conjugates

A blocking assay was developed to understand inhibition. The tested samples are presented in the Table 1. In this assay, F18+E. coli were first incubated for one hour at room temperature with either the carriers or A6-1 conjugates, while gently shaking. Concentrations used were chosen so that the actual A6-1 concentration was 12 μg/ml for A6-1 conjugated carriers CMCM and CMCL (and the similar concentration of non-conjugated carriers) or 120 μg/ml for the A6-1 conjugated carrier polylysine (A6-1-PL-14). These concentrations were chosen because inhibition of binding of F18+E. coli to villi was seen for the unconjugated carriers in the previous tests. Subsequently, fluorescein conjugated A6-1 monomers (A6-F) were added (1 mg/ml) for one hour at room temperature. The fluorescing oligosaccharide was labeled at the reducing end of the molecule with fluorescein (also named 6A1-FITC). Next free A6-1-fluorescein was washed away with PBS followed by centrifugation at 2000 g for 5 min and resuspensions of the pellet with PBS. This was repeated twice.

If carriers without A6-1 bind to the bacteria, they will block the binding of A6-F and no fluorescence-bound bacteria can be detected by fluorescence microscopy. If there is no binding of the carrier to the bacteria in the first step of the test, fluorescing bacteria will be seen. As positive and negative blocking controls, monomeric A6-1 (1 mg/ml) and PBS were used, respectively.

A second way to look at the specificity of the binding of the conjugates and carriers was to look at the effect of washing of bacteria, pre-incubated with a conjugate or carrier, on adhesion to the villi. Washing occurred by centrifugation of the bacteria followed by resuspending them in PBS. If binding to the bacteria was not specific, it was expected that the unbound conjugate and/or carrier were removed during these steps. Following incubation of the washed bacteria with the F18R positive villi, adhesion of bacteria to the villi was counted and the inhibition of bacterial adhesion was calculated as earlier described.

### 2nd Part—In Vivo Testing

Different tests and studies were carried out with the Polylysine-conjugated oligosaccharides as specified in Parts A-E and as defined in Table 1. This A6 based conjugate is also named, hereafter and in the corresponding Figures, Coligo, A6PL or Coligo A6PL.

Part A—In Situ Activity of Coligo A6PL in an In Vivo Small Intestinal Segment Perfusion Model (SISP)

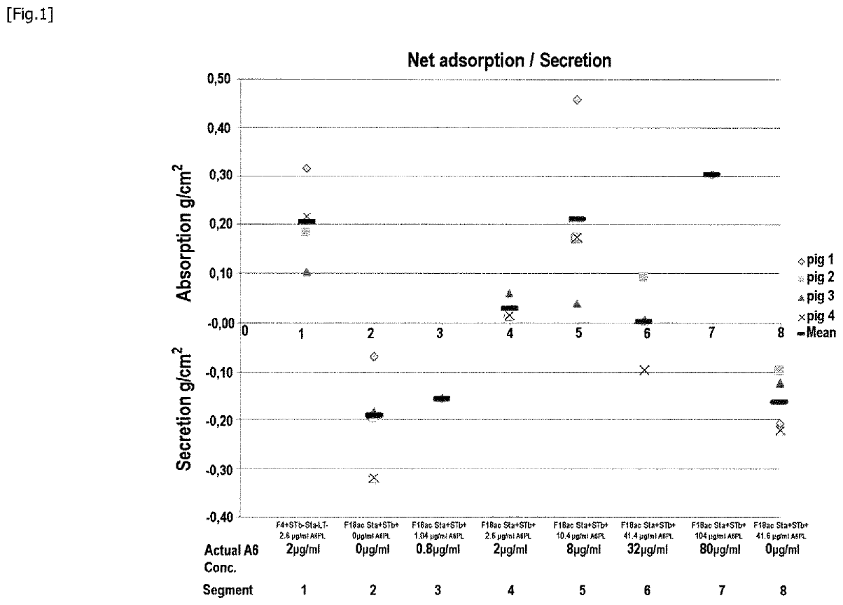

F18R+, F18 seronegative pigs were selected. To select F18R+ pigs, PCR amplification of the F18R linked FUT1 gene on isolated DNA of blood leukocytes was performed as described by Meijerink et al., 1997. Absence of F18 specific serum antibodies was performed with an indirect ELISA (Verdonck et al., 2002). Four F18R+ and F18 seronegative piglets were used to test F18⁺ E. coli inhibition of Coligo A6PL in the in vivo small intestinal segment perfusion model (SISP) (Loos et al., 2013). Pigs were put under anesthesia and 6 segments of jejunum (around 20 cm) were constructed so that fluid could be injected at one site and contents could be collected at the other site of each segment. The following fluids were injected in a segment selected at random:
A negative control (using GIS26 STa− STb− LT−)—segment 1 on FIG. 1,
A positive control (only F18ac bacteria 2134P (STa⁺ STb⁺) and PBS, no conjugate)—segment 2 on FIG. 1,
Three segments for different concentration of Coligo A6PL (A6-1-PL-14) with F18ac bacteria (2134P, the enterotoxigenic strain)
One segment for ungrafted polylysine (PL) and F18ac bacteria 2134P—segment 8 on FIG. 1.
   The table 2 below shows the different conditions in the 6 loos of all 4 piglets.

TABLE 2

| | | Concentration | Concentration | | | | |
|---|---|---|---|---|---|---|---|
| | | Coligo A6PL | A6-1 | Pig | Pig | Pig | Pig |
| | Description | μg/ml | μg/ml | 1 | 2 | 3 | 4 |
| Negative | GIS26 F4⁺ STa⁻STb⁻ LT⁻ + | 2.6 | 2 | X | X | X | X |
| control | Coligo A6PL | | | | | | |
| Positive | 2134P + PBS | 0 | 0 | X | X | X | X |
| control | | | | | | | |
| Tested | 2134P + Coligo A6PL | 1.04 | 0.8 | X | | | |
| product | | | | | | | |
| Tested | 2134P + Coligo A6PL | 2.6 | 2 | | X | X | X |
| product | | | | | | | |
| Tested | 2134P + Coligo A6PL | 10.4 | 8 | X | X | X | X |
| product | | | | | | | |
| Tested | 2134P + Coligo A6PL | 41.6 | 32 | | X | X | X |
| product | | | | | | | |
| Tested | 2134P + Coligo A6PL | 104 | 80 | X | | | |
| product | | | | | | | |
| Tested | 2134P + PL | 41.6 PL | 0 | X | X | X | X |
| product | | | | | | | |

First, 5 ml ungrafted polylysine (PL) or Coligo A6PL or PBS (it depends on the segment) were injected and incubated for 15 min. Next, 5 ml bacteria (8*10⁸ bacteria/mi) was added. The experiment ran for 6 h and every 15 min, 2 ml perfusion fluid (0.9% NaCl+0.1% glucose) were injected. Outcome fluid was collected and the weight was measured. Each segment was measured for the length and wide to calculate the intestinal surface area (cm²). The net absorption was calculated as: (input-output)/surface area (g/cm²). In addition, villi of each pig were collected and used in the in vitro villus adhesion test with 2134P bacteria to confirm the F18R status. Furthermore, at the end of experiment a small section (about 2 cm) was excised from the middle of each loop to be stained with monoclonal anti-FedA antibodies to visualize the colonization of F18⁺ E. coli bacteria in the segment.

Part B—Efficacy of an A6-1-Polylysine Conjugate (Coligo A6PL) Against F18⁺ Reference E. coli Strain Infection

To investigate the in vivo efficacy of Coligo A6PL, a challenge infection experiment was performed in newly weaned piglets. Pig feed or water was supplemented with A6PL and pigs were experimentally infected. Four groups (6 pigs/group, 3-4 week-old, FUT1 GG (F18R⁺), F18 sero-negative) were involved and kept in different stables:
Group 1: +Product supplied via drinking water: 1.6 μg A6-1 (or 2.16 μg Coligo A6PL, A6-1-PL 14) per ml H₂O. Consuming estimation: 2.5 liter H₂O per day per pig (4 mg A6-1 or 5.39 mg Coligo A6PL per pig per day).
Group 2: +product supplied via feed: 8 μg A6-1 (or 10.79 μg Coligo A6PL, A6-1-PL 14) per gram feed. Consuming estimation: 400 gram feed per day per pig (3.2 mg A6-1 or 4.315 mg Coligo A6PL per pig per day).
Group 4: +Control: 2.16 μg polylysine per ml H₂O supplied via drinking water and 21.58 μg polylysine per gram feed (the same concentration in drinking water, high dose in feed, but only polylysine).

Starting from day −3 (D-3), piglets received supplemented feed or water during a 18-day period. At day zero, a challenge infection was performed by intragastrical administration of F18⁺ E. coli strain 107/86 Stx2e positive (10¹¹ CFU per 10 ml) and this was repeated day 1. From day 0 on, excretion of F18⁺ E. coli was monitored by plating faecal samples. Furthermore, animals' weights were followed every week and the antibody responses upon infection were assessed by sampling blood weekly from day −3 on and at euthanasia.

The Table 3 presents the Schematic overview of in vivo challenge trial to investigate the in vivo efficacy of Coligo A6PL.

TABLE 3

| −3 | −2 | −1 | 0 | 1 | 2 | 3 | 4 | 5 | 6 | 7 | 8 | 9 | 10 | 11 | 12 | 13 | 14 | 15 | Day |
|---|---|---|---|---|---|---|---|---|---|---|---|---|---|---|---|---|---|---|---|
| P | P | P | P | P | P | P | P | P | P | P | P | P | P | P | P | P | P | P | Products |
| W | | | | | | | W | | | | | | | W | | | | W | Weight |
| B | | | | | | | B | | | | | | | B | | | | B | Blood |
| | | | F | F | F | F | F | F | F | F | F | F | F | F | F | F | | F | Feces |
| | | | C | C | | | | | | | | | | | | | | | Challenge |
| | | | | | | | | | | | | | | | | | | | Infection |

Part C—1^{St} Field Trial with Coligo A6PL on a Farm with an F18ab⁺STa⁺ Stx2⁺ E. coli (STEC) Infection

The farm was a farm with problems of edema disease.

Four groups of piglets were involved, two control groups (C1 and C2, piglets of different sows) and two groups (P1 and P2) treated with 10.8 mg Coligo A6PL (A6-1-PL 15) in 1 kg feed for 14 days from weaning on. Pigs in the group C1 and C2 are siblings (littermates) of the ones in P1 and P2, respectively. Therefore, statistical analysis compared groups pairwise (C1 versus P1 and C2 versus P2). Each group had 24 pigs, equal in number of males and females. They all expressed the intestinal F18 receptor (F18R+) as characterized by PCR of FUT1 gene (GG/AG). During the trial, weight and feed intake were monitored to calculate the average daily weight gain (ADG), average daily feed intake (ADFI) and feed conversion ratio. The immune response was monitored by determining the F18-specific serum IgA titres. For all animals in the trial, clinical symptoms and mortality were supervised. It is important to note that except for the 10 pigs per group that were selected for observation, the farmer vaccinated all other pigs in each group (14 pigs) against Shiga toxin with ECOPORC SHIGA (IDT Animal Health). This vaccine suppresses the clinical signs of edema disease, but not the bacterial excretion. The table 4 summarizes the intervention activities at different time points during the trial.

TABLE 4
Scheme of 2^{nd} field trial on farm

| d 0 | d 2 | d 4 | d 6 | d 8 | d 10 | d 12 | d 14 | d 16 | d 18 | d 20 | d 22 | d 41 | Time point |
|---|---|---|---|---|---|---|---|---|---|---|---|---|---|
| x | x | x | x | x | x | x | x | Z | | Z | Z | | A6PL feed |
| x | | | | | | | x | | x | | | x | Weight |
| x | | | | x | | | | | x | | | | Blood |

Part D—2^{nd} Field Trial with Coligo A6PL on a Farm with F18ac⁺ STa⁺ LT⁺ E. coli Infection

On this farm an infection with an F18ac⁺ STa⁺ LT⁺ E. coli infection was diagnosed, which is an enterotoxigenic E. coli strain responsible for postweaning diarrhoea. Therefore this farm was selected for a 2^{nd} trial. Similar to the previous trial pigs were assigned to three groups: two control groups (C3 and C4, piglets of different sows), one treatment groups (P3, with A6-1-PL 15). Each group had 22 piglets infected with F18ac⁺ STa⁺ LT⁺ E. coli, equal gender distribution. Here, the treatment was already before weaning, namely from 3 days before weaning till 14 days after weaning. Because treatment before weaning has to be administered to whole litters since piglets suckle their mother, pigs of a litter could not be split over the control and the treatment groups and were not sibling anymore. Whole litters had to be assigned to a control or a treatment group. Therefore, all groups are treated as randomized in the statistical analysis.

All parameters were monitored exactly as in the 1^{st} trial, namely the average daily weight gain (ADG), feed intake (FI), feed conversion ratio (FCR), diarrhea incidence (of 10 pigs/group), anti-F18 IgA response and mortality.

Part E—Effects of Coligo A6PL Active Substance on Performance and Gut Health of Weanling Pigs Experimentally Infected with a Pathogenic E. coli.

Animals, Housing, Experimental Design, and Diet

The protocol for this experiment were reviewed and approved by the Institutional Animal Care and Use Committee at the University of California, Davis. The experiment was conducted at the Cole A at the University of California, Davis. A total of 48 weanling pigs (crossbred; initial body weight (BW): 7.23±1.14 kg) with equal number of barrows and gilts were used in this experiment. The sows and piglets used in this experiment did not receive E. coli vaccines, antibiotic injections, or antibiotics in creep feed. Before weaning, feces were collected from sows and all their piglets included in this study to verify the absence of β-hemolytic E. coli. The F18 E. coli receptor status was also tested in the piglets based on the methods of Kreuzer et al. (2013). All pigs used in this study were susceptible to F18 E. coli. After weaning, all pigs were randomly assigned to one of four dietary treatments in a randomized complete block design with body weight within sex and litter as the blocks and pig as the experimental unit. There were 12 replicate pigs per treatment.

Pigs were individually housed (pen size: 0.61×1.22 m) in environmental control rooms at the Cole facility at University of California, Davis for 19 days, including 7 days before and 11 days after the first E. coli challenge (d 0). The piglets had ad libitum access to feed and water. Environmental enrichment was provided for each pig. The light was on at 0700 and off at 1900 h daily in the environmental control rooms.

The 4 dietary treatments included: 1) Positive control: control diet; 2) AGP: control diet plus 50 mg/kg carbadox (antibiotic); 3) Low dose Coligo (A6-1-PL 19): control diet plus 10 mg/kg Coligo active substance expressed by kg of the administered control diet; and 4) High dose Coligo (A6-1-PL 19): control diet plus 20 mg/kg Coligo active substance expressed by kg of the administered control diet.

Spray-dried plasma and high levels of zinc oxide exceeding recommendation and normal practice were not included in the diets. The experimental diets were fed to pigs throughout the experiment. All diets were formulated to meet pig nutritional requirements (Tables 5-7, NRC, 2012) and provided as mash form throughout the experiment.

TABLE 5
Ingredient compositions of experimental diets¹

| Ingredient, % | Control diet |
|---|---|
| Corn | 44.51 |
| Dried whey | 15.00 |
| Soybean meal | 14.00 |
| Fish meal | 10.00 |
| Soy protein concentrate | 7.00 |
| Lactose | 6.00 |
| Soybean oil | 2.00 |
| Limestone | 0.56 |
| L-Lysine•HCl | 0.15 |
| DL-Methionine | 0.06 |
| L-Threonine | 0.02 |
| Salt | 0.40 |
| Vit-mineral, Sow 6² | 0.30 |
| Total | 100.00 |

¹Three additional diets were formulated by adding 5.5 g/kg Mecadox 2.5 (50 mg/kg Carbadox), 10 mg/kg Coligo, or 20 mg/kg Coligo to the control diet, respectively.
²Provide the following quantities of vitamins and micro minerals per kilogram of complete diet: Vitamin A as retinyl acetate, 11,136 IU; vitamin D3 as cholecalciferol, 2,208 IU; vitamin E as DL-alpha tocopheryl acetate, 66 IU; vitamin K as menadione dimethylprimidinol bisulfite, 1.42 mg; thiamin as thiamine mononitrate, 0.24 mg; riboflavin, 6.59 mg; pyridoxine as pyridoxine hydrochloride, 0.24 mg; vitamin B12, 0.03 mg; D-pantothenic acid as D-calcium pantothenate, 23.5 mg; niacin, 44.1 mg; folic acid, 1.59 mg; biotin, 0.44 mg; Cu, 20 mg as copper sulfate and copper chloride; Fe, 126 mg as ferrous sulfate; I, 1.26 mg as ethylenediamine dihydriodide; Mn, 60.2 mg as manganese sulfate; Se, 0.3 mg as sodium selenite and selenium yeast; and Zn, 125.1 mg as zinc sulfate.

TABLE 6
Calculated energy and nutrient

| | |
|---|---|
| Metabolizable energy, kcal/kg | 3,487 |
| Net energy, kcal/kg | 2,615 |
| Crude protein, % | 22.97 |
| Ile,³ % | 0.86 |
| Leu,³ % | 1.68 |
| Lys,³ % | 1.35 |
| Met,³ % | 0.44 |
| Thr,³ % | 0.79 |
| Trp,³ % | 0.23 |
| Val,³ % | 0.95 |
| Met + Cys,³ % | 0.74 |
| Ca, % | 0.80 |
| Total P, % | 0.69 |
| Digestible P, % | 0.47 |

³Amino acids are indicated as standardized ileal digestible AA.

TABLE 7
Analyzed nutrients, %

| | |
|---|---|
| Dry matter | 89.6 |
| Crude protein | 22.58 |
| Acid Detergent Fiber | 2.87 |
| Neutral detergent fibre | 6.99 |
| Calcium | 1.04 |
| Phosphorous | 0.70 |

Pigs were housed in individual pens for 19 days, including 7 days before and 11 days after the first E. coli challenge. After 7 days adaptation, all pigs were orally inoculated with 3 mL F18 E. coli/day for 3 consecutive days from d 0 post-inoculation (PI). The F18 E. coli were isolated from a field disease outbreak by the University of Illinois Veterinary Diagnostic Lab (isolate number: U.IL-VDL #05-27242). The F18 E. coli expressed heat-labile toxin, heat-stable toxin b, and shiga-like toxins. The inoculums were prepared by the Western Institute for Food Safety and Security at the University of California, Davis and were provided at 1010 CFU per 3 mL dose in phosphate buffer saline (PBS). This dose caused mild diarrhea.

Clinical Observations and Sample Collections

The procedures for this experiment were adapted from the methods of Liu et al. (2013) and Kim et al. (2019a,b). Diarrhea score of each pig was assessed visually each day by 2 independent evaluators, with the score ranging from 1 to 5 (1 normal feces, 2=moist feces, 3=mild diarrhea, 4=severe diarrhea, and 5=watery diarrhea). The frequency of diarrhea was calculated as the percentage of the pig days with a diarrhea score 4 or greater. Pigs were weighed on weaning day, d 0, 5, and 11 PI. Feed intake was also recorded throughout the experiment. Average daily gain (ADG), average daily feed intake (ADFI), and feed efficiency (gain:feed) was calculated for each interval from d −7 to 0, d 0 to 5 PI, and d 5 to 11 PI.

Fecal samples were collected from the rectum of all pigs throughout the experiments using a fecal loop or cotton swap on d 2, 5, 8, and 11 PI to test for β-hemolytic coliforms and percentage (Liu et al., 2013; Kim et al., 2019a,b). Twenty-four pigs (3 barrows and 3 gilts from each treatment) were euthanized on d 5 PI near the peak of infection, and the remaining pigs were euthanized at the end of the experiment (d 11 PI) that was the recovery period of the infection. The selection of necropsy time was based on the results of clinical observations and immune response parameters that were reported in previously published research using same E. coli strain and inoculation dose (Kim et al., 2019a,b).

Before euthanasia, pigs were anesthetized with a 1-mL mixture of 100 mg telazol, 50 mg ketamine, and 50 mg xylazine (2:1:1) by intramuscular injection. After anesthesia, intracardiac injection with 78 mg sodium pentobarbital (Vortech Pharmaceuticals, Ltd., Dearborn, MI) per 1 kg of BW was used to euthanize each pig. Three 3-cm segments from the duodenum, the middle of the jejunum, and the ileum (10 cm close to the ileocecal junction) were collected and fixed in Carnoy's solution. The fixed intestinal tissues were stained with high iron diamine and alcian blue and were analyzed for cross-sectional area of sulfo- and sialomucin, the number of goblet cells per villus, villi height, and crypt depth as described by Almeida et al. (2013) and Kim et al. (2019b). Intestinal contents from distal colon were collected on d 5 and 11 PI when pigs were euthanized for gut microbiome (Mon et al., 2015) and metabolites analysis.

Blood samples were collected from the jugular vein of the pigs (24 pigs with 6 pigs/treatment) that stayed throughout the experiment with or without EDTA to yield whole blood and plasma, respectively, before E. coli challenge (d 0), and on d 2, 5, and 11 PI. Whole blood samples were used for measuring total and differential blood cell count by complete blood count (CBC) test. Serum samples were analyzed for a pro-inflammatory cytokine (TNF-α) and acute phase proteins (C-reactive protein and haptoglobin) using commercial ELISA kits. Serum samples from d 0 before inoculation, d 5 PI and 11 PI (n=6) were also analyzed for targeted metabolites by West Coast Metabolomics Center.

Mesenteric lymph nodes were aseptically collected and then pooled within pig, ground, diluted and plates on brain heart infusion agar for measurement of total bacteria and the results were expressed as CFU per g of lymph node (Almeida et al., 2013; Garas et al., 2016). Spleen samples were analyzed in the same fashion.

Detection of β-Hemolytic Coliforms

Briefly, fecal samples were plated on Columbia Blood Agar with 5% sheep blood to identify hemolytic coliforms, which can lyse red blood cells surrounding the colony. Fecal samples were also plated on MacConkey agar to enumerate total coliforms. Hemolytic colonies from the blood agar were sub-cultured on MacConkey agar to confirm that they were lactose-fermenting bacteria and flat pink colonies. All plates were incubated at 37° C. for 24 h in an air incubator. Populations of both total coliforms and β-hemolytic coliforms on blood agar were assessed visually, with a score from 0 to 8 (0=no bacterial growth, 8=very heavy bacterial growth). The ratio of scores of β-hemolytic coliforms to total coliforms was calculated. Questionable colonies were sub-sub-cultured on new MacConkey and blood agar plates to verify if they were β-hemolytic E. coli by using triple sugar iron agar and lysine iron agar and to verify if they were F-18+E. coli using PCR.

Intestinal Morphology

The fixed intestinal tissues were stained with high iron diamine and alcian blue as described by Almeida et al. (2013). Fifteen straight and integrated villi and their associated crypts and surrounded area were selected to analyze villi height, crypt depth, the number of goblet cells per villus, and cross-sectional area of sulfo- and sialomucin as described by Kim et al. (2019b).

Determination of Total and Differential Blood Cell Counts

Whole blood samples were used to measure total and differential blood cell counts by Comparative Pathology Laboratory at the University of California, Davis. A multiparameter, automated programmed hematology analyzer (Drew/ERBA Scientific 950 FS Hematological Analyzer, Drew Scientific Inc., Miami, FL) was used for the assay to optimally differentiate porcine blood.

Quantitative Real-Time PCR

Total RNA were extracted from jejunal and ileal mucosa samples that were collected on d 5 and 11 PI as in previously described (Liu et al., 2014; Kim et al., 2019b). The RNA quality and quantity were assessed by Agilent Bioanalyzer 2100 (Agilent, Santa Clara, CA). First-strand cDNA was produced from 1 μg of total RNA per sample with SuperScript III First-Strand Synthesis SuperMix for quantitative real time-PCR (qRT-PCR) kit (Invitrogen; Carlsbad, CA) in a total volume of 20 μL. The mRNA expression of Claudin 1 (CLDN1), Interferon gamma (IFNG), Mucin 2 (MUC2), Occludin (OCLN), and Zonula occludens-1 (ZO-1) in jejunal mucosa and the mRNA expression of Interleukin 1 beta (IL1B), Interleukin 6 (IL6), Cyclooxygenase 2 (PTGS2), and Tumor necrosis factor alpha (TNF) in ileal mucosa were analyzed by qRT-PCR. Data normalization was accomplished using beta-actin (ACTB) and Glyceraldehyde 3-phosphate dehydrogenase (GAPDH) as housekeeping genes. Primers were designed based on published literature and commercially synthesized by Integrated DNA Technologies, Coralville, IA. The qRT-PCR reaction conditions followed previously published research (Kim et al., 2019b). The 2-ΔΔCT method was used to analyze relative expression of genes compared with negative control.

Analysis of Gut Microbiome

To determine the impact of Coligo active substance on the relative abundance of microbial populations in fecal samples, 16S rRNA marker gene sequence analysis (V4 region; primers 515F and 806R) was carried out following the procedures from Mon et al. (2015). Bacterial DNA was isolated from gut contents of all individuals using the ZR Fecal DNA MicroPrep Kit (Zymo Research) and polymerase chain reaction (PCR) amplified with a barcoded forward primer as previously described (Mon et al., 2015). Amplicons were pooled and purified with a Qiagen PCR purification column and submitted to the UC Davis DNA Technologies Core for 250 bp pair-end sequencing using the Illumina MiSeq platform. Downstream filtering and bioinformatics analyses were carried out using the open source program Quantitative Insights Into Microbial Ecology (QIIME2) and significant differences in populations via LEfSe (Mon et al., 2015). The data yielded information on how the relative abundance of bacteria along the length of the gastrointestinal tract varies in response to diet and challenge and if there are any difference in alpha and beta diversity.

Metabolome Analysis

Metabolomics analysis was conducted with colon digesta collected on d 5 and 11 PI, and serum samples collected on d 0 before E. coli inoculation, d 5 and 11 PI to determine if supplementing Coligo active substance impacted local metabolites. Six biological replicates from each treatment were submitted to the West Coast Metabolomics Center Advanced Services Core at UC Davis for analysis using both liquid chromatography/mass spectrometry (LC/MS) and gas chromatography/time of flight mass spectrometry (GC/TOF) analysis. The acquired data were processed with partial least squares projection to latent structures and discriminant analysis (PLS-DA). The one-way ANOVA (including all groups) and pairwise-comparison were conducted to identify which compounds were responsible for the difference between dietary groups, the parameters of variable importance projection (VIP)>1, fold change >1.5, and P<0.05 (threshold) were used as criteria.

Transcriptome Analysis

Transcriptome analyses of ileal mucosa from 4 pigs/treatment on d 5 and 11 PI were carried out. Total RNA samples were subjected to an rRNA depletion step with oligodT capture beads and cDNA libraries with a size distribution peak at approximately 300 bp prepared using the Kappa Stranded mRNA-Seq Kit (Kappa Biosystems) with Illumina-compatible barcoded adaptors (IDT). Libraries were pooled into random groups of 6 samples and submitted to the UC Davis Genome Center DNA Technologies Core for 100 bp single read sequencing on the Illumina HiSeq3000/4000 (6 samples/lane). Reads were processed, aligned to the reference genome (Sscrofa 10.2) and preliminary expression analysis performed using CLC Genomics Workbench software (version 8.5.1) to generate means for each group (RPKM), fold change and false discovery rate (FDR)-corrected p-values using the Empirical analysis of DGE tool (exact test for two group comparisons).

Statistical Analysis

Normality of data was verified and outliers were identified using the UNIVARIATE procedure (SAS Inst. Inc., Cary, NC). Outliers were identified and removed as values that deviated from the treatment mean by more than 3 times the interquartile range. Data were analyzed by ANOVA using the PROC MIXED of SAS (SAS Institute Inc., Cary, NC) in a randomized complete block design with the pig as the experimental unit. The statistical model included diet as the main effect and block as random effect. Treatment means were separated by using the LSMEANS statement and the PDIFF option of PROC MIXED. Contrast statements were used to analyze the dose effects of Coligo. The Chi-square test was used for analyzing frequency of diarrhea. Statistical significance and tendency was considered at P<0.05 and 0.05≤P<0.10, respectively.

### Results

### 1st Part—In Vitro Testing

Inhibition of Binding of F18+E. coli by Sugar Mono- and Multimers

1. Comparing Pectin, CMCL, CMCM and Polylysine for Inhibiting Binding of F18+E. coli to Villi

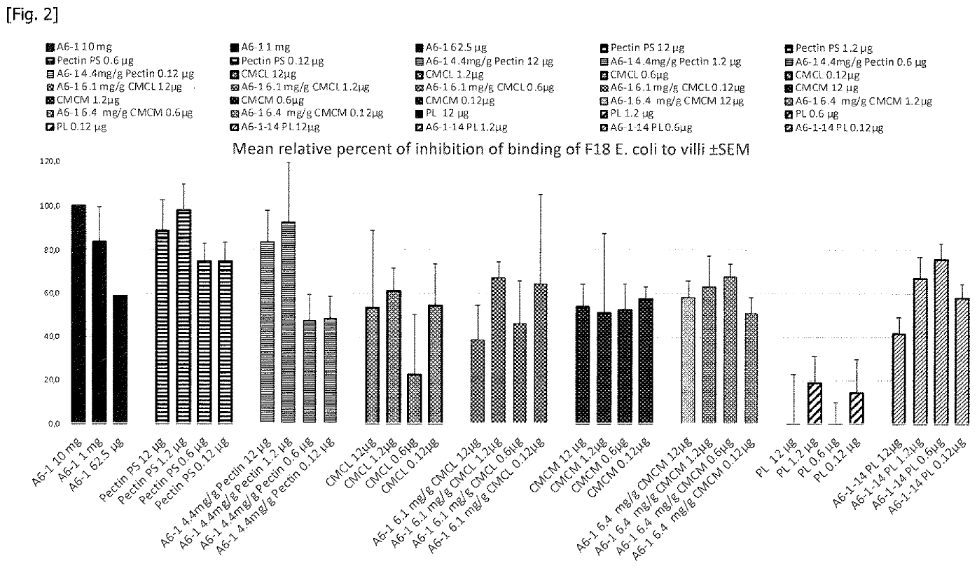

Results on A6-1 conjugated to pectin polysaccharide, CMCL, CMCH and polylysine are given in the tables 8A-D below for the actual A6-1 concentrations of 12, 4.4, 1.2, 0.6, and 0.12 μg/ml. For comparison, results with 10 mg/ml monomeric A6-1 and free carrier resp. are also given in the tables below and a comparison for several conjugates and oligosaccharide monomers is also shown in FIG. 2.

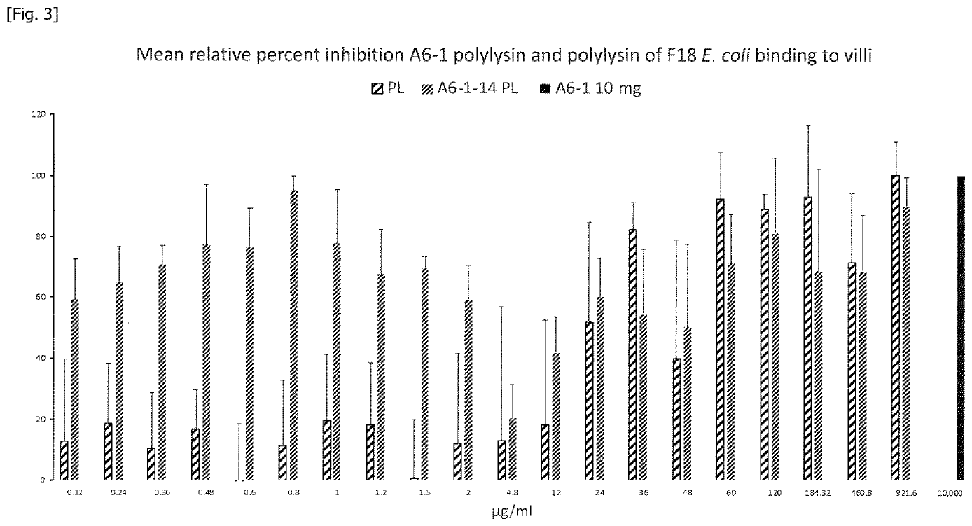

In the tables below it can be seen that pectin, CMCM and CMCL did not show a clear and consistent improved inhibition of binding for A6-1 conjugated carrier in comparison with the unconjugated carrier. Only for polylysine, low concentrations of the multimer showed a good inhibition of binding which reached even 66.6 percent for an actual concentration of A6-1 of 0.8 μg/ml, whereas the carrier at this concentration showed almost no inhibition (8% inhibition). However, when concentrations of the polylysine increased inhibition of binding by the free carrier also increased (Table 8-D below and FIG. 3).

TABLE 8-A
A6-1 conjugated to Pectin polysaccharide

| | | Average | |
|---|---|---|---|
| | Actual A6-1 | % | |
| Description | Concentration | inhibition | SD (%) |
| Free A6-1 monomers | 10 mg/ml | 71.3 | 10.9 |
| Pectin 2.5 mg/ml | 0 μg/ml | 61.6 | 7.2 |
| A6-conjugated pectin 2.5 mg/ml | 12 μg/ml | 59.3 | 9.2 |
| Pectin 1 mg/ml | 0 μg/ml | 61.9 | 32.9 |
| A6-conjugated pectin 1 mg/ml | 4.8 μg/ml | 54.0 | 22.5 |
| Pectin 0.25 mg/ml | 0 μg/ml | 68.2 | 6.2 |
| A6-conjugated pectin 0.25 mg/ml | 1.2 μg/ml | 63.6 | 30.8 |
| Pectin 0.125 mg/ml | 0 μg/ml | 53.6 | 14.6 |
| A6-conjugated pectin 0.125 mg/ml | 0.6 μg/ml | 29.5 | 11.6 |
| Pectin 0.025 mg/ml | 0 μg/ml | 52.8 | 7.3 |
| A6-conjugated pectin 0.025 mg/ml | 0.12 μg/ml | 40.7 | 20.2 |

TABLE 8-B
A6-1 conjugated CMCL lower (type 1) and higher (type 2)
conjugation efficiency (1^{st} part)

| | Actual A6-1 | Average | |
|---|---|---|---|
| CMCL type 1 | Concentration | % inhibition | SD (%) |
| Free A6-1 monomers | 10 mg/ml | 69.5 | 15.4 |
| CMCL 1.97 mg/ml | 0 μg/ml | 42.8 | 10.2 |
| A6-CMCL 1.97 mg/ml | 12 μg/ml | 8.0 | 26.4 |
| CMCL 0.79 mg/ml | 0 μg/ml | 50.4 | 10.3 |
| A6-CMCL2 0.79 mg/ml | 4.8 μg/ml | 44.1 | 16.5 |
| CMCL 0.197 mg/ml | 0 μg/ml | −15.0 | 64.3 |
| A6-CMCL 0.197 mg/ml | 1.2 μg/ml | 45.0 | 26.7 |
| CMCL 0.098 mg/ml | 0 μg/ml | 30.8 | 11.4 |
| A6-CMCL 0.098 mg/ml | 0.6 μg/ml | 44.5 | 26.4 |
| CMCL 0.0197 mg/ml | 0 μg/ml | 9.0 | 82.2 |
| A6-CMCL 0.0197 mg/ml | 0.12 μg/ml | 67.3 | 11.3 |

TABLE 8-B
A6-1 conjugated CMCL lower (type 1) and higher
(type 2) conjugation efficiency (2^{nd} part)

| | Actual A6-1 | Average | |
|---|---|---|---|
| CMCL type 2 | concentration | % inhibition | SD (%) |
| Free A6-1 monomers | 10 mg/ml | 57.05 | 15.42 |
| CMCL 263 μg/ml | 0 μg/ml | 28.32 | 30.43 |
| A6-CMCL 263 μg/ml | 12 μg/ml | 24.92 | 22.25 |
| CMCL 105 μg/ml | 0 μg/ml | 20.17 | 40.59 |
| A6-CMCL 105 μg/ml | 4.8 μg/ml | 31.85 | 21.03 |
| CMCL 26.3 μg/ml | 0 μg/ml | 33.69 | 6.65 |
| A6-CMCL 26.3 μg/ml | 1.2 μg/ml | 37.17 | 3.48 |
| CMCL 13.2 μg/ml | 0 μg/ml | 14.87 | 32.62 |
| A6-CMCL 13.2 μg/ml | 0.6 μg/ml | 27.15 | 23.15 |
| CMCL 2.6 μg/ml | 0 μg/ml | 32.93 | 21.88 |
| A6-CMCL 2.6 μg/ml | 0.12 μg/ml | 35.88 | 39.53 |

TABLE 8-C
A6-1 conjugated CMCM with a lower and higher conjugation
efficiency (1^{st} part)

| | Actual A6-1 | Average | |
|---|---|---|---|
| CMCM type 1 | Concentration | % inhibition | SD (%) |
| Free A6-1 monomers | 10 mg/ml | 63.6 | 6.0 |
| CMCM 1.88 mg/ml | 0 μg/ml | 67.7 | 25.7 |
| A6CMCM 1.88 mg/ml | 12 μg/ml | 78.9 | 12.5 |
| CMCM 0.75 mg/ml | 0 μg/ml | 17.7 | 30.9 |
| A6CMCM 0.75 mg/ml | 4.8 μg/ml | 50.9 | 66.1 |
| CMCM 0.188 mg/ml | 0 μg/ml | −7.3 | 37.6 |
| A6CMCM 0.188 mg/ml | 1.2 μg/ml | 50.4 | 16.6 |
| CMCM 0.094 mg/ml | 0 μg/ml | 60.5 | 23.3 |
| A6CMCM 0.094 mg/ml | 0.6 μg/ml | 38.1 | 59.1 |
| CMCM 0.019 mg/ml | 0 μg/ml | 8.2 | 26.5 |
| A6CMCM 0.019 mg/ml | 0.12 μg/ml | 30.8 | 25.4 |

TABLE 8-C
A6-1 conjugated CMCM with a lower and higher
conjugation efficiency (2^{nd} part)

| | Actual A6 | Average | |
|---|---|---|---|
| CMCM type 2 | concentration | % inhibition | SD (%) |
| Free A6-1 monomers | 10 mg/ml | 61.26 | 13.35 |
| CMCM 262 μg/ml | 0 μg/ml | 33.71 | 17.28 |
| A6-CMCM 262 μg/ml | 12 μg/ml | 34.65 | 3.71 |
| CMCM 104.8 μg/ml | 0 μg/ml | 35.34 | 3.93 |
| A6-CMCM 104.8 μg/ml | 4.8 μg/ml | 58.38 | 15.34 |
| CMCM 26.2 μg/ml | 0 μg/ml | 36.41 | 40.50 |
| A6-CMCM 26.2 μg/ml | 1.2 μg/ml | 40.19 | 21.59 |
| CMCM 13.1 μg/ml | 0 μg/ml | 33.91 | 18.49 |
| A6-CMCM 13.1 μg/ml | 0.6 μg/ml | 42.47 | 14.03 |
| CMCM 2.6 μg/ml | 0 μg/ml | 36.04 | 12.93 |
| A6-CMCM 2.6 μg/ml | 0.12 μg/ml | 32.22 | 13.85 |

TABLE 8-D
A6-1 conjugated polylysines with a lower and higher
conjugation efficiency

| | Percent inhibition of E. coli 107/86 adherence | | |
|---|---|---|---|
| Actual A6-1 | | A6- | A6- |
| Concentration | | POLYLYSINE | POLYLYSINE |
| μg/ml | POLYLYSINE | (A6-1-PL-7) | (A6-1-PL-14) |
| 0.12 | 9.0 | 21.8 | 41.3 |
| 0.24 | 12.9 | 21.4 | 45.4 |
| 0.36 | 7.3 | 33.9 | 49.3 |
| 0.48 | 11.9 | 21.6 | 54.2 |
| 0.6 | -2.1 | 13.4 | 53.7 |
| 0.8 | 8.0 | 24.3 | 66.6 |
| 1 | 13.6 | 14.9 | 54.3 |
| 1.2 | 12.7 | 18.2 | 47.2 |
| 1.5 | 0.5 | 13.6 | 48.6 |
| 2 | 8.4 | 17.7 | 41.4 |
| 4.8 | 9.3 | 13.8 | 14.2 |
| 12 | 11.2 | −5.1 | 29.3 |
| 24 | 36.1 | | 42.0 |
| 36 | 57.5 | | 38.1 |
| 48 | 27.9 | | 35.1 |
| 60 | 64.7 | | 49.8 |
| 120 | 62.4 | | 56.8 |
| 184.3 | 65.2 | | 48.0 |
| 460.8 | 49.9 | | 48.0 |
| 921.6 | 70.1 | | 63.0 |

2. Investigation of Non-A6 Based Inhibition

As aforementioned, high non-AG based inhibition of most carriers was observed. A blocking assay was developed to understand this phenomenon. In this blocking assay, F18+ E. coli were first incubated with either the carriers or AG-conjugated multimers. Subsequently, fluorescence conjugated AG-1 monomers (AG-1-F) were added and binding of the fluorescing AG-1 to the bacteria was determined using a fluorescence microscope.

Incubation with the negative control, PBS, resulted in strong fluorescence of the bacteria, whereas incubation with the positive blocking control A6-1 completely abolished fluorescence. Very weak fluorescence could be seen using following incubation with the carriers CMCL and CMCM or their A6-1-conjugated multimers. No difference was observed in fluorescence between incubation with these carriers and their conjugated constructs, indicating that binding of the carriers to the bacteria occurred. In contrast, a high fluorescence intensity was seen when bacteria were incubated with the unconjugated polylysine, whereas incubation with the A6-1-polylysine multimer almost completely inhibited fluorescence. These results seem to indicate that A6-polylysine bound specifically to F18+ E. coli, whereas the inhibiting activities of A6-CMCL type 2 and A6-CMCM type 2 were purely dependent on the interaction of the carriers (CMCL and CMCM) with the bacteria.

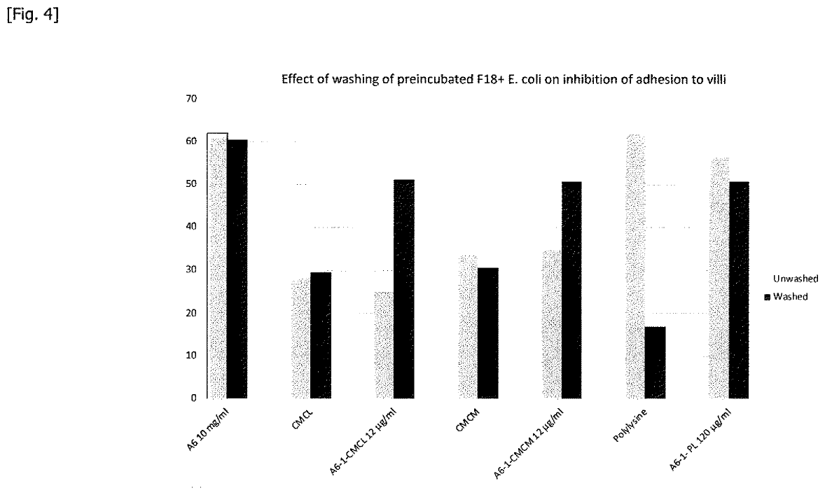

Although only a low non-A6-1 based inhibition was seen for low concentrations of polylysine (FIG. 3), increasing the concentrations of polylysine gave a high non-specific inhibition, so that there was no difference in inhibition by carrier and multimer at actual concentrations of A6-1 in the multimer of ≥30 μg/ml. To understand this, another test was performed. After pre-incubating the bacteria with the conjugates or carriers, bacteria were not directly added to the villi, but were first centrifuged and subsequently washed with PBS twice to remove unbound conjugates. By washing the bacteria the percentage inhibition by polylysine dropped from 62.4% without washing to 16.8% with washing, whereas there was little difference for washed versus unwashed A6-polylysine (FIG. 4). The non-A6 based inhibition decreased significantly upon washing. In contrast, that is not the case for CMCL and CMCM. Since it is known that polylysine could bind to eukaryotic cells (Mazia et al., 1975), we hypothesized that in the unwashed inhibition test, unbound polylysine/A6-1-polylysine adheres to villi, subsequently blocking the binding of bacteria to villi resulting in a non-A6-1 based inhibition. This blocking increases when a high concentration polylysine is used. Because this aspecific blocking occurs using unwashed bacteria, it would also apply to other bacteria which can adhere to the villi. Indeed, when F4⁺ E. coli (GIS26) were used in the in vitro inhibition test, the aspecific blocking also occurred. Interestingly, here A6-1-polylysine showed also this aspecific blocking, but washing completely removed it. This supports our hypothesis that high concentrations of A6-1-polylysine could also block non-specifically the binding of bacteria to the villi, when washed the inhibition most likely is A6-1 specific. Specific binding is more important to prevent or reduce infection in vivo.

3. Inhibition of Binding of F18+E. coli to Villi by Multimers of Blood Group A6-1 Conjugated to Polylysine, Bovine Serum Albumin or Human Serum Albumin

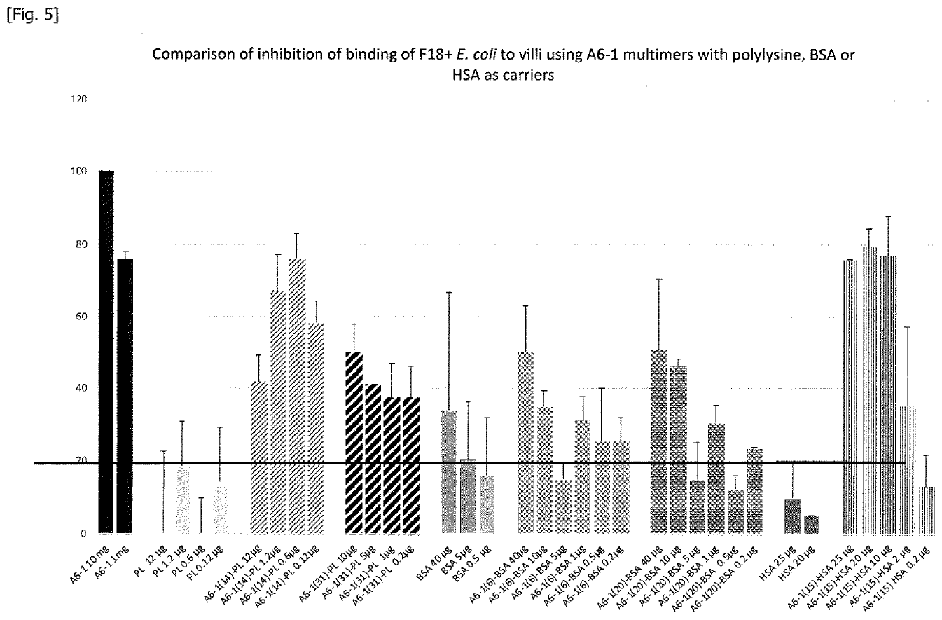

Conjugating blood group A6 type I oligosaccharides on polylysine, BSA or HSA resulted also in inhibition of binding of F18+E. coli to villi of F18R+ pigs. As can be seen in FIG. 5, low actual concentrations of A6-1 conjugated to polylysine most consistently reduced binding of F18+E. coli to villi. The conjugation rate of 14 moles A6-1 per mole polylysine seems more optimal, but rates up till 31 are also functional. Conjugation to HSA also reduced binding, but not when the actual concentration of the blood group sugar A6-1 reached 0.2 μg/ml. At that concentration the polylysine conjugates were still active. BSA-6-1 conjugates were not sufficiently and not consistently effective for actual concentrations of A6-1 of 5 μg/ml and lower.

The results, also obtained with conjugates with alginate and methacrylate carriers, are summarized in Table 9.

TABLE 9
Optimal inhibitory dose range (μmol/ml) of conjugate (in vitro;
inhibitory dose of A6 monomer = at 1000 μg/ml)

| | Conjugate | | | Carrier | | | |
|---|---|---|---|---|---|---|---|
| | inhibition | A6* | Carrier* | inhibition | | A6/C ratio | |
| Conjugate | (M) | (μg/ml) | (mg/ml) | (C)° | M − C°° | (mg A6/g C) | |
| A6-1-PL-7 | 34% | 0.36 | 0.0013 | 7% | 27% | 7 | (612) |
| A6-1-PL-14 | 67% | 0.8 | 0.001 | 8% | 59% | 14.7 | (768) |
| CMCL type 1 | 45% | 1.2 | 0.197 | −15% | 45% | 0.3 | (6.1) |
| CMCM type 1 | 50% | 1.2 | 0.188 | −7.3% | 50% | 1 | (6.4) |
| Pectin | 64% | 1.2 | 0.25 | 68% | −5% | na | (4.8) |
| Alginate | 41% | 0.6 | 0.081 | 56% | −15% | na | (7.4)) |
| Metacrylate | 71.3% | 1.2 | 1 | 71% | 0.3% | na | (3) |

*supplied via the conjugate
°carrier only without A6
°°the difference M − C is a measure of the specific blockage of F18 fimbriae by A6

4. Inhibition of Binding of F18+E. coli to Villi Using Multimers of Different Oligosaccharides Conjugated to Polylysine

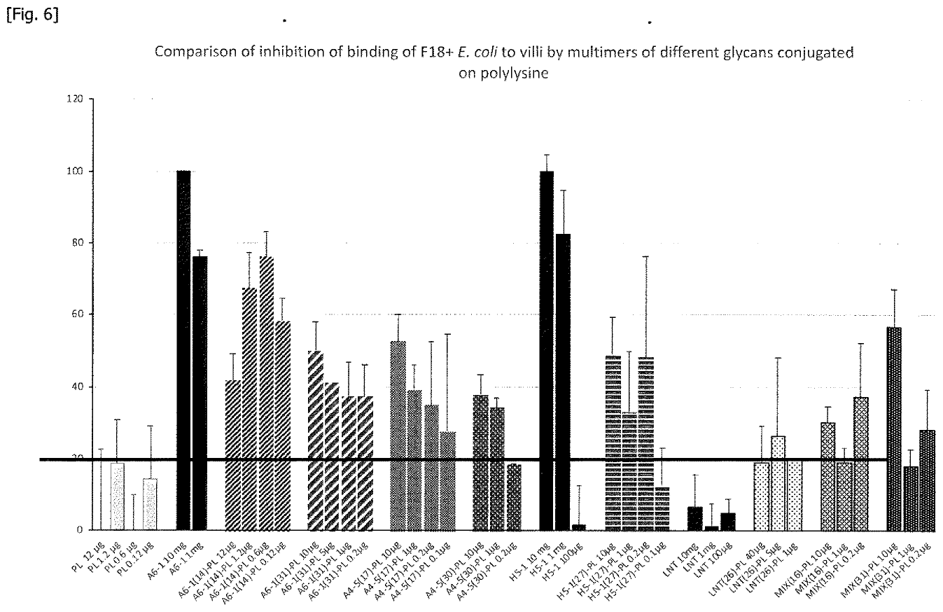

Different blood group sugars were conjugated to polylysine (FIG. 6). The conjugate of polylysine with A6-1 showed the best inhibition of binding of F18⁺ E. coli to villi for very low actual concentrations of A6-1 as compared to conjugates comprising other types of carriers. In addition, conjugates of the other two tested blood group sugars (A4-5 and H5-1) with polylysine showed also between 30 and 40% inhibition for actual concentrations of the sugars of 10 to 0.2 μg/ml. Lacto-N-tetraose, a control sugar, did not do this. Conjugating a more crude mix of A6-1, formed during the production process, resulted in a less efficient reduction of adhesion.

### 2^{nd} Part—In Vivo Testing

Results of Part A—In Situ of Coligo A6PL Activity in an In Vivo Small Intestinal Segment Perfusion Model (SISP)

The FIG. 1 depicts the in situ activity of Coligo A6PL in the segment perfusion test. As negative control (osmotic control), segments incubated with enterotoxigenic depleted GIS26 (number 1) showed net adsorption of perfusion fluid (±0.2 g/cm²). In contrast, as positive control, segments incubated with PBS first and then the F18+ bacteria (number 2) secreted 0.19 g/cm² (−0.19 g/cm²). Bacteria colonized the intestine and elaborated enterotoxins which induced secretion. When the segments were treated with different concentration of Coligo A6PL before addition of F18+ bacteria, the same concentration dependent inhibition was observed as seen in vitro: 2 μg/ml resulted in net adsorption (+0.03 g/cm²) (number 4) and almost complete prevention of fluid los was seen at 8 μg/ml (+0.21 g/cm²) (number 5). This effect decreased using 32 μg/ml (0 g/cm²) (number 6) whereas the same concentration of polylysine (number 8) could not prevent secretion (−0.16 g/cm²). Also when a higher concentration of Coligo A6PL was used (80 μg/ml actual A6 concentration), secretion of fluid was completely abolished and fluid adsorption was obtained (±0.3 g/cm²) (number 7). This result is probably caused by the coating effect of polylysine on the villi as demonstrated in the in vitro inhibition assay.

Results of Part B—Efficacy of Against F18⁺ Reference E. coli Strain Infection

1. Survival and Weight Gain

Upon infection, pigs did not show clinical symptoms. Eating and drinking were normal in all groups. On day 6, one pig from the control group (1 per 6 animals) deceased with clinical symptoms of edema. This mortality ratio (1 on 6) is in concordance with mortality ratio seen in natural infections. One pig from the drinking water group deceased, but not due to the F18 E. coli infection.

When the trial started, pigs in the feed supplemented group (i 7.9 kg) were slightly heavier than in the other groups (7.2-7.5 kg). During the experiment, the drinking water and the control group showed similar weight gain whereas the feed group grew a bit faster. One pig died due to edema disease in the control group (ratio 1/6).

2. Coligo A6PL Interferes with F18⁺ E. coli Colonization and Reduces the Shedding Duration

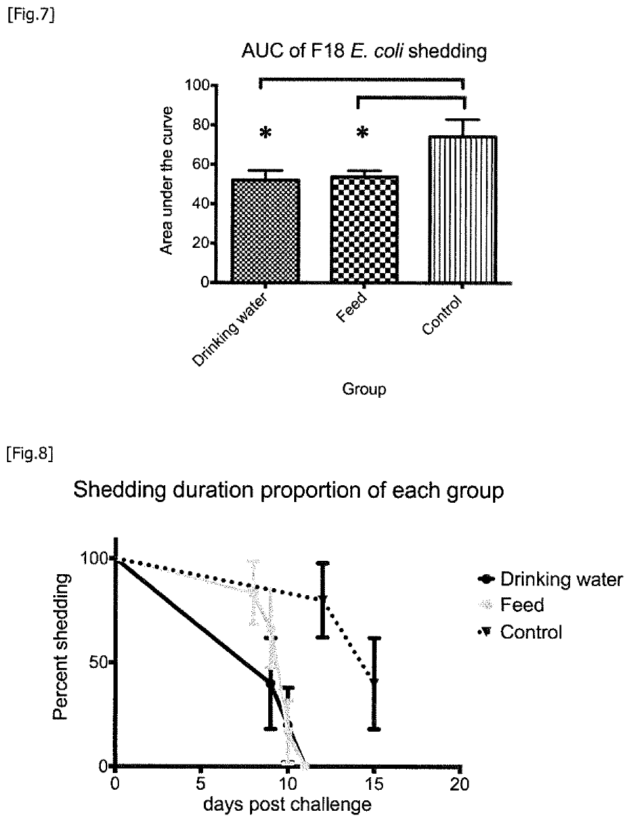

Excretion of F18+E. coli by pigs was monitored daily from the inoculation till 13 days after the first infection as well as the day they were euthanized (day 15). All pigs had stopped excreting F18+E. coli in their feces at day 15, except for 2 pigs of the control group. From one of these two pigs, bacteria were also detected in jejunum and ileum at euthanasia, suggesting that these pigs could excrete the bacteria longer. The individual shedding curves for each pig were used to calculate area under the curve (FIG. 7). The area (log 10 number of F18+E. coli excretion per gram feces×15 days) represents the effect of the treatment on the colonization of F18⁺ E. coli in the gut during the whole 15-day duration. Area under the curve of each treatment group was compared to the area under the curve of the control group using the Mann-Whitney U test. Drinking water group and feed group were significantly different from the control group (P-value=0.03 for both).

Since each pig stopped shedding at different time points, the median shedding duration (days) of each group was calculated and was 9, 10, 10 and 15 days for the drinking water, the low dose, the high dose and the control group, respectively. Furthermore, shedding durations of the treated groups were compared with this of the control group using Gehan-Breslow-Wilcoxon test with an adjusted significant level set at 0.016 (=0.05/3 comparisons). Shedding duration of drinking water and feed group were significantly shorter than this of the control (p=0.0035 and 0.0034, respectively). These results indicate that A6-1-polylysinePL interfered with F18+E. coli colonization; thereby shortening the shedding duration (FIG. 8).

3. Immune Response Upon Challenge with F18⁺ E. coli

Upon challenge, bacteria colonize the gut and animals develop immunity against the pathogens, Using Coligo A6PL as anti-adhesive analog, interference with F18+E. coli colonization occurs which could influence the immune response against the pathogen. Therefore the serum antibody response was determined. Within each group, there was a significant difference between the anti-F18 IgG level at day −3 (day starting product treatment) and this at day 15. In the feed group, the IgG at day 11 was also significantly higher than this at day −3. Between the treated groups and the control, no significant difference in IgG was seen for all time points although IgG was lower in the feed group and the drinking water group at day 11 than for the other groups. Similarly, at day 15, the IgG of feed group was lowest but not significant.

IgA responses are necessary for mucosal immunity of the host against the F18+E. coli in the gut. Most of the serum IgA is induced in the gut reflecting the local antibody response. Comparably to the anti-F18 IgG response, anti-F18 IgA were for each group significantly lower at day −3 than at day 11 and day 15. However, there was no significant difference for the IgA between groups at the different time points although the IgA at day 15 was lower in the drinking water group and the feed group than the control group; Similarly, the F18-specific IgA level at day 11 was lowest in the drinking water group but was not significantly different from the other groups.

These results demonstrate that all groups showed an antibody response to the F18⁺ E. coli, meaning that the pathogen still contacted the mucosa sufficiently enough in the treatment groups to activate the immune system. Although no significant differences were seen between groups at d11 and d15, the drinking group and the feed group showed lower responses, which is in accordance with results of faecal shedding, and is additional evidence that in these two groups contact between the pathogen and the mucosa was less than in the other groups.

In conclusion, Coligo A6PL could not prevent colonization of bacteria into the gut but it reduces the adhesion of the pathogen and protects pigs from mortality. Consequently, excretion duration is shortened and there is enough time for the animals to build up immunity against the pathogens. This again demonstrates the effectiveness of supplementing Coligo A6PL via drinking water or feed.

Results of Part C—1^{st} Field trial with Coligo A6PL on a farm with an F18ab⁺STa⁺ Stx2e⁺ E. coli (STEC) infection

1. Average Daily Weight Gain (ADG)

Measurement of weight at days 0, 8, 18 and 41 allowed to calculate the average daily weight gain over 3 periods: (1) the period of treatment with the Coligo A6PL product from weaning till day 14, (2) from day 14 till day 18 and (3) from day 18 till day 42 (before the fattening period). Piglets in the treated groups grew better than the controls during the period of treatment, but not significant (P-value: 0.46 (C1 vs P1) and 0.18 (C2 vs P2)). After this period, pigs in all groups were catching up weight and similar weight gain compared between the sibling groups (C2 vs P2). After this period, pigs in all groups were catching up weight and similar weight gain compared between the sibling groups.

2. Feed Intake (FI) and Feed Conversion Ratio (FCR)

FI and FCR ratio were monitored during the period of treatment of the product (day 0 till day 14). Pigs in the product groups are a bit less but grow a bit more than their siblings. Therefore, the FCRs are also different between paired groups: C1 was clearly higher than P1 and C2 was clearly higher than P2 during oligosaccharide supplementation (Table 10). So looking over the groups the supplementation resulted in 220 g less food consumption per kg pigs grew. However, since the unit for which this is calculated in the trial is the group, no statistics could be performed on these data in this trial. Nevertheless, results indicated increasing economic performance upon using Coligo A6PL.

TABLE 10
Feed intake (FI) and feed conversion ratio (FCR) of each group
during the treatment period with Coligo A6PL in the farm A

| | Total | | | FCR |
|---|---|---|---|---|
| | consuming | FI0-14 | ADG0-14 | (kg feed/ |
| Group | (kg) | (g) | (g) | kg body) |
| C1 (Control) | 105 | 312.5 | 228.0 | 1.37 |
| C2 (Control) | 68 | 202.4 | 122.3 | 1.65 |
| P1 (+PL-A6) | 98.7 | 293.8 | 243.8 | 1.21 |
| P2 (+PL-A6) | 65.8 | 195.8 | 148.8 | 1.32 |

3. Immune Response Against F18⁺ E. coli

A parameter which could visualize the small intestinal colonization of the pathogens was the serum IgA antibody response against F18 fimbriae. These fimbriae bind to receptors on small intestinal enterocyte brush borders and allow the pathogen to be in close contact with the mucosa, and release their toxins which are transported over the mucosa in the circulation to reach target cells. This close contact allows the pathogen to grow on the mucosal surface. As a result, the mucosal immune system of the pig will become activated and the induction of a mucosal F18 specific IgA antibody response will be initiated. Approximately 70% of this IgA will be secreted in the small intestine, where it can neutralize the pathogen, the other 30% diffuses in the serum, where it can be detected as a measure of the small intestinal colonization.

The anti-F18 IgA titer was measured in sera of all pigs at 3 different time points: at weaning (day 0), at 8 and at 18 dpw (day post weaning). Animals showing increasing anti-F18 IgA titers at 8 and/or 18 dpw as compared to day 0 were considered as responding. Out of 24 pigs, 18 in C1, 15 in P1, 14 in C2 and 18 in P2 responded with an IgA response against F18⁺ E. coli, indicating there was no difference in the number of responding pigs comparing treated groups with their sibling group (P-value=0.359). However, comparing the titers of the responding pigs in treated versus control groups differences were seen. IgA responding animals in group P2 had a significantly lower titer at d8 (P-value=0.002) reflecting a lower colonization during the preceding period. The IgA titer of the responding animals in treated group P1 at d8 was similar as this of the sibling control, but significantly dropped at 18 dpw (P-value=0.006) suggesting a different colonization profile of shorter duration than in the sibling control. Anti-F18 IgA responses in both control groups were similar and reflect a normal long colonization.

In conclusion, the 1^{st} field trial shows as positive results of the A6PL supplementation namely: (1) a decreased small intestinal colonization with F18⁺ E. coli reflected by a lower serum anti-F18 IgA response at day 8 and 18 in the treated group P2 and (2) an improved feed conversion ratio. Importantly also no side effects of the feed supplementation were observed.

Results of Part D—2^{nd} Field Trial with Coligo A6PL on a Farm with an F18ac⁺ STa⁺ LT⁺ E. coli Infection

1. Average Daily Weight Gain

Pigs were weighed at weaning, on 14, 18 and 42 dpw. This allowed to calculate the ADG during 3 periods: the treatment period (day 0 till 14 dpw), the 2nd period between 14 dpw and 18 dpw and the 3^{rd} period from 18 dpw till 42 dpw. During the treatment period, the treated group P3 showed the highest ADG but not significantly higher than this in the control groups. However, ADG was significantly higher in P3 than in C3 during the second period (P-value=0.03). During the 3^{rd} period no significant difference between groups occurred.

2. Feed Intake and Feed Conversion Ratio

Group P3 had the best conversion efficiency, (Table 11). Statistical analysis could only be performed taking into account data of both trials. This calculation is added at the end.

TABLE 11
Feed intake (FI) and feed conversion ratio (FCR)
in the trial Lannoo

| | | | | FCR |
|---|---|---|---|---|
| | Total consuming | FI | ADG | (kg feed/ |
| Group | (kg) | (g) | (g) | kg body) |
| C3 (Control) | 45.8 | 148.6 | 94.8 | 1.57 |
| C4 (Control) | 48.3 | 156.9 | 103.6 | 1.51 |
| P3 (Product) | 45.2 | 147.8 | 115.2 | 1.28 |

3. Immune Response Against F18⁺ E. coli

A parameter that is related with colonization of the small intestine by the pathogen is the anti-F18 specific serum IgA response, as explained in the result section of the 1^{st} trial. Out of the 22 pigs in each group, 13 in C3, 15 in C4 and 9 in P3 responded against F18. The number of responding animals in the control groups (C3 and C4) is significantly higher than in the treated group (P3) (P-value=0.0328), suggesting a decreased colonization in the treatment group. For pigs which showed an anti-F18 IgA antibody response the titres did not significantly differ between control and treatment.

The conclusion for the 2^{nd} field trial is similar to this of the first trial: there is a significant lower IgA response in the treated groups as compared to the controls, suggesting that the receptor of the Coligo A6PL indeed captures bacteria decreasing the bacterial colonization in the small intestine. Secondly, weight gain and the feed conversion ratio of the treated group P3 is better than these in the control groups, similar to what was seen in the first trial. Treating animals with Coligo A6PL could bring economic benefit: pigs eat less but grow better.

Results of Part E—Effects of Coligo A6PL on Performance and Gut Health of Weanling Pigs Experimentally Infected with a Pathogenic E. coli

The objectives of this experiment were: 1) to investigate the influence of dietary supplementation of Coligo on diarrhea score and growth performance of weanling pigs experimentally infected with a pathogenic E. coli, 2) to determine the effects of dietary Coligo on gut morphology, fecal culture score, and gut microbiome and metabolites of weanling pigs infected with E. coli, and 3) to determine the effects of dietary Coligo on gut barrier function and immunity of weanling pigs infected with E. coli.

Growth Performance and Diarrhea Score

No difference was observed in initial BW (body weight) and d 0 BW of pigs among dietary treatments.

Pigs supplemented with antibiotics had greater (P<0.05) BW on d 5 PI than pigs in the positive control and Coligo A6PL treatments, whereas pigs supplemented with Coligo A6PL had greatest (P<0.05) BW on d 11 PI among all dietary treatments. Supplementation of Coligo A6PL enhanced (P<0.05) ADFI of pigs from d 5 to 11 PI, compared with positive control. Supplementation of Coligo enhanced (P<0.05) feed efficiency from d 0 to 5 PI compared with pigs in the positive control group.

Pigs supplemented with antibiotics, like carbadox, had lowest (P<0.05) average diarrhea score from d 0 to 5 PI and d 5 to 11 PI among all dietary treatments.

Compared with positive control, pigs supplemented with Coligo A6PL reduced average diarrhea score of weaned pigs from d 0 to 5 PI, which was not the case from d 5 to 11 PI. However, supplementation of antibiotics or Coligo A6PL reduced (P<0.05) the frequency of diarrhea of weaned pigs from d 0 to 11 PI.

Intestinal Morphology

On d 5 PI, supplementation of Coligo A6PL dose-dependently increased (linear, P<0.05) villi height, the ratio of villi height to crypt depth, villi width, and villi area in duodenum, enhanced (linear, P<0.05) the ratio of villi height to crypt depth in jejunum, and increased (linear, P<0.05) villi height, the ratio of villi height to crypt depth, and villi area in ileum, compared with the control.

Supplementation of Coligo A6PL also enhanced (linear, P<0.05) duodenal and jejunal villi height and jejunal and ileal villi area, and tended to increase (linear, P<0.10) the ratio of villi height to crypt depth in jejunum and ileal villi height on d 11 PI. Pigs fed with antibiotics had greater (P<0.05) villi height in duodenum and ileum, the ratio of villi height to crypt depth in all three intestinal segments, and villi area in duodenum than pigs in the control group on d 5 PI. On d 11 PI, pigs supplemented with antibiotics also had higher (P<0.05) villi height in all three intestinal segments, greater (P<0.05) villi height to crypt depth ratio in jejunum, and bigger (P<0.05) sialomucin area in duodenum than pigs in the control group. Pigs in the antibiotics group also had greater (P<0.05) villi height:crypt depth in all intestinal segments on d 5 PI, and greater (P<0.05) villi height in ileum, in comparison to Coligo treatments.

Fecal Culture and Bacterial Translocation

Pigs supplemented with antibiotics had lower (P<0.05) percentage of 3-hemolytic coliforms in feces than pigs in the other 3 treatments on d 2 and 5 PI, and had lower (P<0.05) β-hemolytic coliforms in feces than pigs in the positive control on d 8 PI (Table 12).

TABLE 12
The percentage (%) of β-hemolytic coliform
of weaned pigs fed diets supplemented with Coligo

| | Diet | | | | | | | |
|---|---|---|---|---|---|---|---|---|
| | Positive | Low | High | | | P-value | | |
| | Control | Coligo | Coligo | AGP | SEM | Diet | Linear³ | Quadratic³ |
| d 2 PI¹ | 68.86^{a} | 65.85^{a} | 66.25^{a} | 31.56^{b} | 7.97 | <0.01 | 0.76 | 0.83 |
| d 5 PI¹ | 58.97^{a} | 54.43^{a} | 53.11^{a} | 8.79^{b} | 7.77 | <0.01 | 0.41 | 0.81 |
| d 8 PI ² | 17.17^{a} | 12.48^{ab} | 13.05^{ab} | 2.77^{b} | 4.53 | <0.05 | 0.48 | 0.59 |
| d 11 PI ² | 7.17 | 0.00 | 2.18 | 2.10 | 2.28 | 0.16 | 0.13 | 0.07 |

^{a, b}Within a row, means without a common superscript differ (P < 0.05).
¹Each least squares mean represents 11-12 observations.
² Each least squares mean represents 5-6 observations.
³Linear and quadratic effects of adding Coligo to the control diet.

No difference was observed in the percentage of 0-hemolytic coliforms in feces among dietary treatments on d 11 PI. Supplementation of high dose Coligo reduced bacterial translocation in lymph nodes on d 5 and 11 PI, whereas supplementation of low or high dose Coligo, or antibiotics reduced bacterial translocation in spleen on d 11 PI, compared with pigs in the positive control (Table 13).

TABLE 13
Bacterial translocation in lymph nodes and spleen
of weaned pigs fed diets supplemented with Coligo

| | Diet | | | | | | | |
|---|---|---|---|---|---|---|---|---|
| | Positive | Low | High | | | P-value | | |
| Item¹ | Control | Coligo | Coligo | AGP | SEM | Diet | Linear | Quadratic |
| d 5 PI | | | | | | | | |
| Lymph | 307^{a} | 131^{ab} | 63^{b} | 147^{ab} | 57.6 | <0.05 | <0.05 | 0.51 |
| node, cfu/g | | | | | | | | |
| Spleen, | 175 | 106 | 121 | 47 | 58.9 | 0.54 | 0.54 | 0.63 |
| cfu/g | | | | | | | | |
| d 11 PI | | | | | | | | |
| Lymph | 510^{a} | 330^{ab} | 152^{b} | 279^{ab} | 120.2 | 0.09 | <0.05 | 0.99 |
| node, cfu/g | | | | | | | | |
| Spleen, | 1039^{a} | 154^{b} | 94^{b} | 56^{b} | 258.1 | <0.05 | <0.05 | 0.24 |
| cfu/g | | | | | | | | |

^{a, b}Within a row, means without a common superscript differ (P < 0.05).
¹Each least squares mean represents 5 to 6 observations

Fecal Microbiota Changes

Alpha diversity indices of faecal microbiota collected from different time points were determined.

No difference was observed in alpha diversity indices among 4 treatments during the entire experimental period. The majority of indices were increased (P<0.05) on d 5 PI, but decreased (P<0.05) on d 12 PI. There were differences observed in bacterial community composition in fecal samples collected from different time points and differences were also observed in bacterial community composition of fecal samples in pigs in antibiotics group compared with the other treatments on d 11 PI (adjusted P-value <0.05).

The relative abundance of Bacteroidetes and Proteobacteria was decreased (P<0.05), but the relative abundance of Firmicutes and Actinobacteria was increased (P<0.05) in fecal samples of weaned pigs as the age increased. On d 5 PI, supplementation of Coligo tended (P<0.10) to increase Bacteroidetes and reduce Proteobacteria in feces of weaned pigs compared with the control diet, regardless of dose. The top 3 abundant bacterial phyla in fecal samples were Firmicutes, Bacteroidetes, and Proteobacteria. Within Firmicutes phylum, the relative abundance of Peptococcaceae and Ciotridiaceae was increased (P<0.05), but the relative abundance of Lachnospiraceae was decreased (P<0.05) in fecal samples as the age of animal increased. Supplementation of antibiotics increased (P<0.05) the relative abundance of Clostridiaceae and Peprostreptococcaceae, but decreased (P<0.05) the relative abundance of Peptococcaceae and Lactobacillaceae in the fecal samples collected on d 11 PI, in comparison with other dietary treatments. No difference was observed in Firmicutes phylum between control and Coligo treatments. Within Bacteroidetes phylum, the relative abundance of Prevotellaceae was increased (P<0.05), but the relative abundance of Bacteroidaceae and Rikenellaceae was decreased (P<0.05) in fecal samples of pigs when they were older. The relative abundance of Paraprevotellaceae was lower (P<0.05) in pigs supplemented with low Coligo than pigs in the other groups. Within Proteobacteria phylum, Succinivibrionaceae and Enterobacteriaceae were most abundant on d −7, and their abundances were reduced (P<0.05) as pigs getting older. On d 5 PI, pigs supplemented with Coligo has greater (linear, P<0.05) relative abundance of Desulfovibrionaceae, but lower (linear, P<0.05) relative abundance of Succinivibrionaceae in fecal samples compared with control pigs. Supplementation of antibiotics reduced (P<0.05) the relative abundance of Helicobacteraceae but increased (P<0.05) Desulfovibrionaceae in fecal samples of weaned pigs, compared with the control.

Metabolite Profile in Serum

Untargeted metabolomics was applied to assess the metabolite profiles in serum collected from pigs on d 0 (before the inoculation), d 5 PI and at the end of the experiment (d 11 PI). A total of 354 (134 identified and 220 unidentified) metabolites were detected in serum. No significant differences were observed in serum metabolites on d 0.

Three metabolites are involved in glycerolipic metabolism, such as, glycerol, glycerol-alpha-phosphate, and propylene glycol. These metabolites were reduced by antibiotics supplementation. In addition, inosine, hypoxanthine, and guanosine that are involved in purine metabolism were also reduced by antibiotics supplementation. However, hypoxanthine was increased by antibiotics supplementation. There were 2 metabolites related to pyruvate metabolism, including propylene glycol, lactic acid. These changes were confirmed by enrichment analysis, in which glycerolipid metabolism (3 hits, unadjusted P=0.000041585 and FDR=0.0033268) and purine metabolism (3 hits, unadjusted P=0.00099446 and FDR=0.039778) stood out as being significant affected. In addition, galactose metabolism and glycolysis or gluconeogenesis were also in top 5 enrichments that differed among treatments in serum on d 5 PI.

On d 11 PI, the PLS-DA score plot based revealed a distinct partition of antibiotics versus Coligo, however, metabolites in serum of pigs in the control group had overlapped with both antibiotics and Coligo groups. There were 14 identified metabolites significantly differed in serum among dietary treatments (P<0.05; q 0.20; Table 14).

TABLE 14
Metabolite profiles in serum of weaned pigs on d 11 post E.
coli inoculation ¹.

| | Fold change | Fold change | Fold change |
|---|---|---|---|
| | Control vs | Low Coligo vs | High Coligo vs |
| Metabolite | Antibiotics | Antibiotics | Antibiotics |
| 2-hydroxybutanoic acid | 0.552 | NS* | NS |
| allantoic acid | 0.636 | NS | NS |
| alpha-ketoglutarate | 0.65 | NS | NS |
| chenodeoxycholic acid | 3.015 | NS | 2.562 |
| ethanolamine | 0.587 | NS | 0.563 |
| glyceric acid | 0.643 | NS | NS |
| glycerol | 0.332 | NS | NS |
| glycerol-alpha-phosphate | 0.609 | NS | 0.561 |
| guanine | 0.608 | NS | NS |
| hypoxanthine | 0.614 | NS | 0.652 |
| inositol-4-monophosphate | 0.484 | NS | NS |
| linoleic acid | 0.634 | NS | NS |
| pantothenic acid | NS | NS | 0.645 |
| raffinose | NS | 0.518 | NS |
| thymidine | 0.643 | NS | NS |
| thymine | 0.555 | NS | NS |

¹ A cut-off q value (FDR adjusted P-value) less than 0.2 denotes statistical significance.

Three metabolites were involved in glycerolipid metabolism in control versus antibiotics group, including glyceric acid, glycerol, and glycerol-alpha-phosphate, and these metabolites were reduced by antibiotics supplementation. In high Coligo versus antibiotics group, 2 metabolites involved in glycerophospholipid metabolism, including ehanolamine and glycerol-alpha-phosphate, were reduced by antibiotics supplementation. These changes were consistent with enrichment analysis as well, in which glycerolipid metabolism (3 hits, unadjusted P=0.00055807 and FDR=0.044646) and glycerophospholipid metabolism (2 hits, unadjusted P=0.0024812 and FDR=0.19849).

Metabolite Profile in Colon Digesta

Untargeted metabolomics was also applied to assess the metabolite profiles in colon digesta collected from pigs on d 5 and d 11 PI. A total of 398 (178 identified and 220 unidentified) metabolites were detected in colon digesta. Seven identified metabolites in colon digesta on d 5 significantly differed between low Coligo and antibiotics, while 19 identified metabolites differed between high Coligo and antibiotics (P<0.05; q≤0.20; Table 15). However, no significant differences have been observed in further pathway analysis.

TABLE 15
Metabolite profiles in colon digesta of weaned pigs on d 5
post E. coli inoculation.¹

| | Fold change | Fold change |
|---|---|---|
| | Low Coligo vs | High Coligo vs |
| Metabolite | Antibiotics | Antibiotics |
| 2-hydroxyglutaric acid | 3.619 | NS* |
| 2-hydroxyvaleric acid | 11.320 | NS |
| 4-pyridoxic acid | 0.585 | NS |
| alpha-aminoadipic acid | NS | 1.637 |
| arabitol | 2.031 | 3.010 |
| benzoic acid | NS | 1.572 |
| cellobiose | NS | 2.278 |
| conduritol-beta-epoxide | NS | 2.024 |
| cysteine | NS | 2.966 |
| docosahexaenoic acid | NS | 2.428 |
| ferulic acid | NS | 2.186 |
| glycerol | NS | 0.658 |
| indole-3-acetate | NS | 7.972 |
| maltotriose | NS | 4.855 |
| monomyristin | NS | 1.687 |
| octadecylglycerol | 3.320 | 3.314 |
| palmitoleic acid | NS | 1.927 |
| phosphate | NS | 0.323 |
| proline | NS | 2.360 |
| tocopherol acetate | 0.474 | NS |
| tocopherol gamma- | 0.555 | 0.652 |
| tranexamic acid | NS | 12.061 |
| xanthosine | NS | 1.883 |

¹A cut-off q value (FDR adjusted P-value) less than 0.2 denotes statistical significance.

The PLS-DA score plot based on Component 1 and Component 2 revealed a distinct partition among dietary treatments on d 11 PI. There were 23 identified metabolites in colon digesta significantly differed between control and low Coligo, 4 metabolites differed between low Coligo and antibiotics, while 15 identified L metabolites differed between high Coligo and antibiotics on d 11 P (P<0.05; q 0.20; Table 16).

TABLE 16
Metabolite profiles in colon digesta of weaned
pigs on d 11 post E. coli inoculation.¹

| | | | | |
|---|---|---|---|---|
| | | Fold | | |
| | Fold | change | | |
| | change | Low | Fold change | Fold change |
| | Control vs | Coligo vs | Low Coligo | High Coligo |
| | Low | High | vs | vs |
| Metabolite | Coligo | Coliogo | Antibiotics | Antibiotics |

(first part)

| | | | | |
|---|---|---|---|---|
| 1-hexadecanol | 1.961 | NS* | NS | NS |
| 2-hydroxyhexanoic | 0.353 | NS | NS | NS |
| acid | | | | |
| 2-hydroxyvaleric acid | NS | NS | NS | 9.755 |
| 3-hydroxybutyric acid | NS | NS | NS | 2.619 |
| 3,6-anhydro-D- | 0.435 | NS | NS | NS |
| galactose | | | | |
| 3,6-anhydro-D-glucose | 0.393 | NS | NS | NS |
| 4-hydroxybutyric acid | NS | NS | NS | 1.878 |
| 6-deoxyglucose | NS | NS | 2.085 | 2.305 |
| adenosine | 0.398 | NS | 3.204 | NS |
| aminomalonate | NS | NS | NS | 2.030 |
| arabitol | NS | NS | NS | 3.123 |
| arachidonic acid | 0.519 | NS | NS | NS |
| azelaic acid | NS | NS | NS | 2.502 |
| dodecanol | NS | NS | 1.646 | NS |
| fructose | 0.441 | NS | 4.265 | NS |
| fructose-6-phosphate | NS | NS | NS | 0.208 |
| glucose | 0.470 | NS | NS | NS |
| glutamic acid | NS | NS | NS | 1.750 |
| glycine | NS | NS | NS | 1.836 |
| heptadecanoic acid | 2.567 | NS | NS | NS |
| homoserine | NS | NS | NS | 1.846 |
| hydroquinone | 0.186 | NS | NS | NS |
| isoheptadecanoic acid | 3.328 | NS | NS | NS |

(second part)

| | | | | |
|---|---|---|---|---|
| isopentadecanoic acid | 2.664 | NS | NS | NS |
| montanic acid | 1.900 | NS | NS | NS |
| myo-inositol | 0.481 | NS | NS | NS |
| nonadecanoic acid | 2.375 | NS | NS | 1.939 |
| octadecanol | 1.772 | NS | NS | NS |
| oxoproline | NS | NS | NS | 1.630 |
| pentadecanoic acid | 2.584 | NS | NS | NS |
| pyruvic acid | NS | NS | NS | 2.140 |
| phytanic acid | 1.683 | 0.496 | NS | NS |
| pimelic acid | 2.010 | NS | NS | NS |
| threonic acid | 0.555 | NS | NS | NS |
| tranexamic acid | 0.157 | NS | NS | NS |
| urea | 0.314 | NS | NS | NS |
| uric acid | 3.346 | NS | NS | NS |
| xanthosine | NS | NS | NS | 2.020 |

¹A cut-off q value (FDR adjusted P-value) less than 0.2 denotes statistical significance.

Several metabolic pathways, including butanoate metabolism (4 metabolites involved: 3-hydroxybutyric acid, 4-hydroxybutyric acid, glutamic acid, and pyruvic acid), glutathione metabolism (3 metabolites involved: glutamic acid, glycine, and oxoproline), glycine, serine and threonine metabolism (3 metabolites involved: glycine, homoserine, and pyruvic acid), pentose and glucuronate interconversions (3 metabolites involved: arabitol, fructose-6-phosphate, and pyruvic acid), alanine, aspartate and glutamate metabolism (2 metabolites involved: glutamic acid and pyruvic acid), and methane metabolism (2 metabolites involved: fructose-6-phosphate and glycine) have been significantly affected in high Coligo versus antibiotics.

These changes were confirmed by enrichment analysis, in which, butanoate metabolism (4 hits, unadjusted P=0.000042024 and FDR=0.0033619), glutathione metabolism (3 hits, unadjusted P=0.00093148 and FDR=0.037259), glycine, serine and threonine metabolism (3 hits, unadjusted P=0.0018508 and FDR=0.049352), pentose and glucuronate interconversions (3 hits, unadjusted P=0.0024676 and FDR=0.049352), alanine, aspartate and glutamate metabolism (2 hits, unadjusted P=0.006952 and FDR=0.11123), and methane metabolism (2 hits, unadjusted P=0.013706 and FDR=0.18275).

### Discussion

E. coli diarrhea is one of top 3 prevalent diseases in pre-weaning and post-weaning pigs, which is responsible for anorexia, slower growth, even death of pigs. Results of the present studies indicate that supplementation of the conjugates as provided herein improved growth rate, reduced frequency of diarrhea and systemic inflammation of weaned pigs experimentally challenged with F18 E. coli

In the current study, pigs supplemented with Coligo or antibiotics had reduced frequency of diarrhea, indicating both Coligo substance and antibiotics enhanced disease resistance of weaned pigs. In consistence with clinical signs' results, pigs supplemented with antibiotics had less β-hemolytic coliforms compared with pigs in the control on d 8 PI, whereas Coligo supplemented pigs had intermediate low β-hemolytic coliforms among dietary treatments. This observation indicates pigs supplemented with Coligo or antibiotics either had lower F18 E. coli shedding in their feces throughout the infection period or they excluded F18 E. coli out of their intestine quicker than pigs in the positive control. Take altogether, either way could help pigs in both treatments recovered sooner from E. coli infection.

Tight junction is critical to control both intracellular and paracellular permeability and to prevent leaky gut. Several multiprotein junctional complexes are responsible for tight junction, including zonulae occludens (ZO), occludins, and claudins. In the present study, supplementation of high dose of Coligo enhanced the mRNA expression of ZO1 and OCLN at the peak of E. coli infection, and supplementation of low dose Coligo still had relatively high CLDN1 expression on d 11 PI (results not shown), compared with control pigs. No difference was observed in gene expression of tight junction proteins in pigs fed Coligo and antibiotics. These results indicate that supplementation of Coligo could enhance gut integrity of weaned pigs, which was supported by the intestinal morphology analysis, as indicated by bigger villi height and villi height to crypt depth ratio, and greater villi area. This was probably also attributed to the decreased bacterial translocated to lymph nodes and spleen. Although bacterial translocation happens naturally and continuously in the gastrointestinal tract of an animal, however, most bacteria are killed during the passage or in the lymph organs by immune cells. Therefore, mesenteric lymph nodes and other organs are sterile in a healthy and immunocompetent animal. Results of tight junction protein gene expression, bacterial translocation, and intestinal morphology suggest that pigs in Coligo and antibiotics groups may have better intestinal health, which was responsible for better nutrient digestion and absorption and hence better performance.

The colonized F18 E. coli could produce large amount toxins, such as heat-labile toxin, heat-stable toxins, and lipopolysaccharides. Those endotoxins trigger the synthesis of cytokines and acute phase proteins, followed by local and systemic inflammation. Cytokines produced by many different cell types may have different activities regulating inflammation, although they also share overlapping activities. Based on previously published research with the same bacteria strain, F18 E. coli infection could induce systemic inflammation, such as increasing white blood cell counts, neutrophils, and lymphocytes, as well as enhancing pro-inflammatory cytokine and acute phase protein concentration in serum of weaned pigs (Song et al., 2012; Liu et al., 2013). In the present study, pigs supplemented with Coligo have lower numbers of white blood cell, neutrophils, and lymphocytes on d 2 PI, and lower neutrophils on d 5 P1. In addition, pigs in low dose or high dose Coligo groups had reduced serum C-reactive protein and haptoglobin. These observations indicate that pigs in Coligo groups had less severe systemic inflammation compared with pigs in the control group. Consistently, it was also observed that a relative low mRNA expression of inflammatory markers (i.e., IL1B, IL6, and TNF) in ileal mucosa of weaned pigs fed with Coligo. Take altogether, results of this experiment indicate that pigs in Coligo and antibiotics groups had enhanced disease resistance, therefore had less severe systemic and local inflammation caused by E. coli infection.

Chao 1 index measures microbial population richness and Shannon index measures microbial diversity of gut microbiota (Chao, 1984; Shannon, 1948). Results of alpha-diversity indicated that the age of pig has more impact on fecal microbiota diversity throughout the 18-day experiment, compared with dietary treatments. Supplementation of antibiotics significantly altered the bacterial community composition in feces by increasing the relative abundance of Clostridiaceae, Peptostreptococcaceae, and Desulfovibrionaceae, but decreasing the relative abundance of Helicobacteraceae, Peptococcaceae, and Lactobacillaceae. However, supplementation of Coligo increased the relative abundance of Desulfovibrionaceae, but decreased the relative abundance of Paraprevotellaceae and Succinivibrionaceae compared with control. Interestingly, pigs in the Coligo groups had greater relative abundance of Lactobacillaceae than pigs in the antibiotics group. Species from Lactobacillaceae have promising probiotic properties to improve overall health and disease resistance and are well accepted as probiotics. Results of the current study indicated supplementation of Coligo also modified gut microbiome and may enhance the beneficial bacteria diversity in weaned pigs infected with E. coli. This modification may be related to the better performance and intestinal health of pigs supplemented with Coligo. Results of gut microbiome analysis also indicated the different microbiota modification by Coligo versus antibiotics.

Metabolomics analysis indicated that supplementation of antibiotics had more impacts on the metabolite profiles of serum, especially at the peak of E. coli infection, d 5 PI. A clear separation was observed in serum metabolites of pigs between low Coligo and antibiotics on d 5 PI. The most impacted metabolic pathways by antibiotics included glycerolipic metabolism and purine metabolism. Again, similar separated pattern was also observed in colon digesta samples on d 11 PI. The most different metabolic pathways between low Coligo and antibiotics included butanoate metabolism, glutathione metabolism, glycine, serine and threonine metabolism, pentose and glucoronate interconversions, and alanine, aspartate and glutamate metabolism. In the present study, supplementation of Coligo, especially low dose Coligo oppositely affected the production of many metabolites in serum and colon digesta in comparison to supplementation of antibiotics. The changed metabolite profiles are likely due to the modified gut microbiome by two different types of supplements.

The results in the present invention indicate that in-feed supplementation of the oligosaccharide conjugates as provided herein enhanced disease resistance of weaned pigs as indicated by reduced frequency of diarrhea during the entire experimental period. Supplementation of the oligosaccharide-conjugates of the invention was shown to enhance growth performance of F18 E. coli infected pigs. These benefits are closely related to three modes of action. First, pigs supplemented with the oligosaccharide-conjugate of the invention had better intestinal integrity than pigs in the control group. Second, pigs supplemented with the oligosaccharide-conjugate of the invention had milder intestinal inflammation and systemic inflammation compared with positive control animals either due to having less severe E. coli infection or enhanced immune responses. Third, supplementation of the oligosaccharide-conjugate of the invention modified gut microbiome and their metabolites in the intestine by enhancing the relative abundance of beneficial microorganisms.

Results of this experiment also indicate that the oligosaccharide-conjugate of the invention and antibiotics may have different impacts on gut microbiome and their metabolites in weaned pigs.

### REFERENCES

Miura, Y., Hoshino, Y., & Seto, H. (2015). Glycopolymer Nanobiotechnology. Chemical Reviews, 116(4), 1673-1692.
Almeida, J. A., Y. Liu, M. Song, J. J. Lee, H. R. Gaskins, C. W. Maddox, O. Osuna, and J. E. Pettigrew. 2013. Escherichia coli challenge and one type of smectite alter intestinal barrier of pigs. J. Anim. Sci. Biotech. 4:52.
Bannerman, D. D. and S. E. Goldblum. 2003. Mechanisms of bacterial lipopolysaccharide-induced endothelial apoptosis. Am. J. Physiol. Cell Mol. Physiol. 284:L899-L914.
Bertschinger H U, Bachmann M, Mettler C, Pospischil A, Schraner E M, Stamm M, Sydler T, Wild P, 1990. Vet Microbiol. 25(2-3):267-81
Chao, A. 1984. Nonparametric estimation of the number of classes in a population. Scand. J. Stat. 265-270.
Cox, E., Houvenaghel. A., 1993. Vet. Microbiol. 34(1), 7-18; Verdonck, F., Cox, E., van Gog, K., Van der Stede, Y., Duchateau, L., Deprez, P., Goddeeris, B. M., 2002. Vaccine 20(23-24), 2995-3004
Garas, L. C., C. Feltrin, M. K. Hamilton, J. V. Hagey, J. D. Murray, L. R. Bertolini, M. Bertolini, H. E. Raybould, and E. A. Maga. 2016. Milk with and without lactoferrin can influence intestinal damage in a pig model of malnutrition. Food Funct. 7:665-678.
Kim, K., A. Ehrlich, V. Perng, J. A. Chase, H. Raybould, X. Li, E. R. Atwill, R. Whelan, A. Sokale, and Y. Liu. 2019a. Algae-derived β-glucan enhanced gut health and immune responses of weaned pigs experimentally infected with a pathogenic E. coli. Anim. Feed Sci. Technol. 248:114-125.
Kim, K., Y. He, X. Xiong, A. Ehrlich, X. Li, H. Raybould, E. R. Atwill, E. A. Maga, J. Jørgensen, and Y. Liu. 2019b. Dietary supplementation of Bacillus subtilis influenced intestinal health of weaned pigs experimentally infected a pathogenic E. coli. J. Anim. Sci. Biotechnol. 10:52-63.
Kreuzer, S., M. Reissmann, and G. A. Brockmann. 2013. New fast and cost-effective test to get the ETEC F18 receptor status in pigs. Vet. Microbiol. 163:392-394.
Liu, Y., M. Song, T. M. Che, J. A. S. Almeida, J. J. Lee, D. Bravo, C. W. Maddox, and J. E. Pettigrew. 2013. Dietary plant extracts alleviate diarrhea and alter immune responses of weaned pigs experimentally infected with a pathogenic Escherichia coli. J. Anim. Sci. 91:5294-5306.
Liu, Y., M. Song, T. M. Che, J. J. Lee, D. Bravo, C. W. Maddox, and J. E. Pettigrew. 2014. Dietary plant extracts modulate gene expression profiles in ileal mucosa of weaned pigs after an Escherichia coli infection. J. Anim. Sci. 92:2050-2062.
Mon, K. K. Z., P. Saelao, M. M. Halstead, G. Chanthavixay, H. Chang, L. Garas, E. A. Maga, and H. Zhou. 2015. Salmonella enterica Serovars Enteritidis infection alters the indigenous microbiota diversity in young layer chicks. Front. Vet. Sci. 2:61-76. NRC. 2012. Nutrient requirements of swine. 11th rev. ed. Natl. Acad. Press, Washington, DC.
Shannon, C. E. 1948. A mathematical theory of communication. Bell system technical journal 27, 379-423.
Song, M., Y. Liu, J. A. Soares, T. M. Che, O. Osuna, C. W. Maddox, and J. E. Pettigrew. 2012. Dietary clays alleviate diarrhea of weaned pigs. J. Anim. Sci. 90:345-360.
Meijerink E, Fries R, Vögeli P, Masabanda J, Wigger G, Stricker C, e al. Two (1,2) fucosyltransferase genes on porcine chromosome 6q11 are closely linked to the blood group inhibitor (S) and Escherichia coli F18 receptor (ECF18R) loci. Mamm Genome 1997; 8:736-41.
F. Verdonck, E. Cox, K. Van Gog, Y. Van der Stede, P. Deprez, L. Duchateau, B. Goddeeris, 2002. Different antibody responses following infection of newly weaned piglets with a F4 enterotoxigenic E. coli strain or a F18 verotoxigenic E. coli strain. Vaccine, 20, 2993-3004.
Loos M., Hellemans A., Cox E., 2013. Optimization of a small intestinal segment perfusion model for heat-stable enterotoxin A induced secretion in pigs. Vet. Imm. Immunopath., 152, 82-86.
Loos M, Geens M, Schauvliege S, Gasthuys F, van der Meulen J, Dubreuil J D, Goddeeris B M, Niewold T, Cox E. Role of heat-stable enterotoxins in the induction of early immune responses in piglets after infection with enterotoxigenic Escherichia coli. PLoS One. 2012; 7(7):e41041.
Tiels P, Verdonck F, Smet A, Goddeeris B, Cox E. The F18 fimbrial adhesin FedF is highly conserved among F18+ Escherichia coli isolates. Vet Microbiol. 2005 Oct. 31; 110(3-4):277-83.
VAN DEN BROECK, W., Cox, E., & Goddeeris, B. (1999). Seroprevalence of F4(+) enterotoxigenic Escherichia coli in regions with different pig farm densities. VETERINARY MICROBIOLOGY, 69(3), 207-216.

1. Conjugates (I) which comprise n saccharidic chains X(Fucα2)Galβk(Y)TV (II) covalently grafted on a linear protein W, wherein:
X is absent, or is Galα3 or GalNAcα3 and when X is absent, then Y is absent;
Y is absent or is Fucα2 or Fucα4;
T is absent or is ZNAcε3;
and wherein Z is Glc or Gal; and ε is α or β;
V is absent or is a mono, or an oligosaccharide;
n is an integer equal to 8 or more,
k is 3 or 4,
and wherein the covalent grafting occurs either between V and the linear protein W, when V is present, or between T and the linear protein W, when T is present and V is absent, or between Galβk and the linear protein W, when both T and V are absent.

2. Conjugates (I) according to claim 1, wherein k is 3.

3. Conjugates (I) according to claim 1, wherein W is a polylysine, in particular a poly-L-lysine, such as ε-poly-L-lysine.

4. Conjugates (I) according to claim 1, wherein the saccharidic chains (II) are covalently coupled on the linear protein, by the saccharidic unit corresponding to their reducing end which is open and acyclic and has been coupled by its aldehyde function to a primary amino function of the linear protein by reductive amination.

5. Conjugates (I) according to claim 1 wherein n is in the range from 8 to 240, particularly from 8 to 40, and in particular in the range from 10 to 35, more specifically in the range from 12 to 35.

6. Conjugates (I) according to claim 1, wherein the saccharidic chains (II) are selected from the group consisting of the following oligosaccharides:
Fucα2Gal;
Fucα2Galβ3GlcNAcβ3Gal;
Fucα2Galβ3GlcNAcβ3Galβ4Glc;
Galα3(Fucα2)Galβ3GlcNAcβ3Gal;
GalNAcα3(Fucα2)Galβ4Glc;
Galα3(Fucα2)Galβ3GlcNAcβ3Galβ4Glc;
GalNAcα3(Fucα2)Galβ4GlcNAcβ3Galβ4Glc;
GalNAcα3(Fucα2)Gal;
GalNAcα3(Fucα2)Galβ3GlcNAcβ3Gal;
GalNAcα3(Fucα2)Galβ3GlcNAcβ3Galβ4Glc;
GalNAcα3(Fucα2)Galβ3(Fucα4)GlcNAcβ3Gal;
GalNAcα3(Fucα2)Galβ3(Fucα4)GlcNAcβ3Galβ4Glc;
Galα3(Fucα2)Galβ3(Fucα4)GlcNAcβ3Gal;
Galα3(Fucα2)Galβ3(Fucα4)GlcNAcβ3Galβ4Glc;
GalNAcα3(Fucα2)Galβ3GlcNAcβ3Galβ3GlcNAcβ3Gal;
GalNAcα3(Fucα2)GalβGlcNAcβ3Galβ3GlcNAcβ3Galβ4Glc;
GalNAcα3(Fucα2)Galβ3GalNAcβ3Galα4Gal;
GalNAcα3(Fucα2)Galβ3GalNAcβ3Galα4Galβ4Glc;
GalNAcα3(Fucα2)Galβ3GalNAcα3(Fucα2)Galβ3GlcNAcβ3Gal and
GalNAcα3(Fucα2)GalβGalNAcα3(Fucα2)Galβ3GlcNAc 3Galβ4Glc.

7. Conjugates (I) according to claim 1, wherein the polylysine, and in particular, the ε-poly-L-lysine, has a weight average molecular weight Mw in the range from 2000 to 33000 g/moL, in particular in the range from 3200 to 6850 g/moL and/or an average degree of polymerization (DP) in the range from 15 to 240, in particular in the range from 20 to 50.

8. A pharmaceutical composition comprising a pharmaceutically acceptable carrier and one or more of the conjugates according to claim 1 in a therapeutically effective amount.

9. A food or drink or pig feed composition supplemented with one or more of the conjugates according to claim 1.

10. Conjugates (I) according to claim 1, for use as a medicament.

11. Conjugates (I) according to claim 1, for use in treating or preventing F18⁺ E. coli infections in mammals, such as pigs, in particular in treating or preventing of a post weaning diarrhea and/or edema disease in pigs.

12. Conjugates (I) for use according to claim 10, wherein the use is on pigs and the conjugate is included in a pig feed composition.

13. Method for preparing a conjugate according to claim 1 wherein, n molecules of saccharidic chains X(Fucα2)Galβk(Y)TV (II), with n, k, X, Y, T and V, are grafted by covalent coupling, via their reduced end on a molecule of a linear protein W.

14. Method according to claim 13 wherein the grafting is obtained by reaction between the oligosaccharide or polysaccharide of formula X(Fucα2)Galβk(Y)TV (II) and the linear protein.

15. Method according to claim 13 wherein the linear protein has pendant primary amino groups, in particular is polylysine, and the grafting on the linear protein is carried out by reaction of reductive amination, between primary amino groups of the linear protein, in particular polylysine, and the aldehyde group of the reducing-end in its acyclic form of the oligosaccharide or polysaccharide of formula X(Fucα2)Galβk(Y)TV (II), with k, X, Y, T and V, in the presence of a reducing agent, such as NaBH₃CN.
